(19) United States
(12) Patent Application Publication
(10) Pub. No.: US 2009/0012432 A1
(43) Pub. Date: Jan. 8, 2009

(54) State Based Birth Monitoring System

(75) Inventor: Yehuda Sharf, Tel-Aviv (IL)

Correspondence Address: MARTIN D. MOYNIHAN d/b/a PRTSI, INC., P.O. BOX 16446, ARLINGTON, VA, 22215, US

(73) Assignee: BARNEV LTD., Netanya (IL)

(21) Appl. No.: 10/599,687

(22) PCT Filed: Apr. 7, 2005
(86) PCT No.: PCT/IL2005/000380

§ 371 Date: Sep. 8, 2008

(51) Int. Cl. A61B 5/103

(52) U.S. Cl. 600/588

(57) ABSTRACT

A method of monitoring a birth process, comprising: receiving, over time, a plurality of position signals from one or more positioning elements or tissue areas located at least one of a cervix and a fetal head; and determining a discrete state of labor of a fetus that is wholly inside a body responsive to said position signals, with a temporal resolution of better than 15 minutes, said discrete state being other than a start or stop of labor and encompassing more than a single contraction, said state including a state other than an abnormal fetal head position.

### RELATED APPLICATIONS

The present application is a 119(e) of U.S. Provisional Application 60/560,291 filed on Apr. 7, 2004 the disclosure of which is incorporated herein by reference. The present application is also a continuation-in-part of PCT/IL2004/001092 filed on Nov. 29, 2004, the disclosure of which is incorporated herein by reference.

### FIELD OF THE INVENTION

The present invention relates to the field of birth monitoring.

### BACKGROUND

Most children are born by vaginal birth, and the vast majority of births at least in western countries, are carried out in a hospital under the monitoring of doctors and/or midwives. Among the main concerns of the medical staff during labor are to assess the progress of labor, to identify problems and complications and suggest and/or perform treatment to overcome these problems and complications as well as preventing or reducing damage to the mother and/or fetuses.

The standard management of labor progress is using as a reference a “partogram”. FIG. 1 is a graph, known as a partogram, showing the average and by inference the “ideal” cervical dilatation and fetal head descent as related to birth progression. According to Friedman, who first described this partogram and is a well-known authority in the field, the labor process is subdivided into phases and divisions with physiologic and clinical importance. Friedman established norms by which a series of dysfunctional labor situations could be defined and detected. The graph illustrates the typical curves for a normal primigravida labor (a woman having her first labor), and a similar graph (not shown) exists for multipara labor (a woman having subsequent labors). The first stage of labor is characterized by dilatation of the Cervical os, first slowly in a latent phase and then faster, in an active phase accompanied with descent of the fetal head. When the Cervical os is fully dilated, the second stage of labor, travel of the fetus along a birth canal, begins.

In the active phase of the first stage of labor, Freidman described (a) an acceleration stage, where the cervical dilatation rate increases, (b) a phase of maximum slope of the rate of dilatation and (c) a phase of deceleration of dilatation (some papers claim that a deceleration phase does not exist and name it a plateau stage, where dilatation is relatively steady). The onset of active labor is commonly defined to begin when the cervix is 3 to 4 cm or more dilated in the presence of uterine contractions. As will be described below, this is used to help classify labor complications.

Friedman developed a concept of three functional divisions: preparatory, dilatational, and pelvic to describe the physiological objectives of each division. However, monitoring the transit from one division to the next requires repeated and frequent manual palpitation (described below), by an experienced physician. Specifically, for example, the onset of the pelvic division (which involves descent of the fetal head from the abdomen to the pelvis and the movement of the fetal head in the pelvis), which commences with the deceleration phase and typically involves engagement, flexion, descent, internal rotation, extension and external rotation, is difficult to identify. In any case, frequent manual palpitation is not only unpleasant and at times painful for the patient but also increases the danger of infection and requires an inordinate amount of time by the physician or other attendant (as compared to the time currently spent managing labor).

While the partogram indicates a statistical norm of the progression of labor, most births vary from this norm. However, many complications can only be identified if and once a major deviation from the norm occurs. In addition, the partogram ignores many variables which might otherwise provide an indication of labor progress/abnormality. U.S. Pat. No. 6,423,016, the disclosure of which is incorporated herein by reference, describes a decision support system which integrates additional information and assists in comparing a current progress with a partogram and ranking the current labor.

The technique commonly used for labor management is manual palpation of the cervix by two fingers inserted through the vagina. This palpation is used to estimate various parameters, such as cervical dilatation, cervical effacement, tissue consistency, fetal head station and position (some of these are commonly recorded and documented). In addition, various rules have been devised to help indicate abnormal situations, for example based on rate of change of one or more monitored parameters. Also, manual palpation can be used to directly detect various pathologies, for example, cephalopelvic disproportion, pelvic inadequacy, asynclitism and changes in shape of the fetal head such as molding or caput succedaneum.

Manual palpation of the cervix has several problems. Palpation is commonly performed only once every 1-4 hours, it is at least partly subjective, inaccurate and highly subjected to intra- as well as inter-observer errors. As noted above, vaginal examination is generally unpleasant and at times painful to the patient, it requires the presence of a skilled operator and may possibly result in contamination of the patient being monitored.

Several publications have suggested attaching sensors to the Cervical os, so its dilatation may be measured mechanically and automatically, without physical examination. For example, U.S. Pat. No. 5,935,061 to Acker et al., the disclosure of which is incorporated herein by reference suggests small wireless position sensors for attachment to a Cervical os and fetal head. This application also suggests that a neural network based system can be used to learn and later detect “retrograde contractions” and “deviant fetal positions”, using positional signals and motion vectors generated using the wireless sensors.

Once the first stage of labor is completed, the fetus starts leaving the uterus and passes along the birth canal. The degree of descent of the fetal head along the birth canal is commonly characterized by “stations” along the canal, which are currently numbered, in a substantially arbitrary manner, as the distance in cm of the fetal head from a line connecting the pelvic Ischial spines. Again, this is estimated manually, with the associated possible disadvantages of manual methods.

Various complications are known to occur in active labor. The following are excerpts from “Williams Obstetrics”, with some changes and remarks added.

Sokol and co-workers reported that 25% of nulliparous labors were complicated by active phase abnormalities while 15% of multigravidas developed this problem. Indeed, active phase disorders are apparently the most common abnormalities of labor. Friedman subdivided active-phase problems into protraction and arrest disorders. Protraction is defined as a slow rate of cervical dilatation or descent:

a. for nulliparas, less than 1.2 cm/hour dilatation or less than 1 cm/hour;

b. for multiparas, less than 1.5 cm/hour dilatation or less than 2 cm/hour descent.

It should be noted that detecting a protraction is by definition a lengthy process and may cause unnecessary problems/complications to mother and/or fetus.

Arrest is a complete cessation of dilatation or descent. Arrest of dilatation was defined as 2 hours with no cervical change, and arrest of descent as 1 hour without fetal descent.

### Abnormal Labor (Dystocia)

Caphelopelvic disproportion (CPD)—obstructed labor due to disparity between the dimensions of the fetal head and the maternal pelvis. Such true caphelopelvic disproportion is rare and most disproportions are due to malposition of the fetal head—(the head being flexed or hyperextended), asynclitism (the head being tilted to side). Rarely is it due to a truly large head or a contracted pelvis. CPD may be relative and a result of ineffective uterine contractions. Inability to achieve vaginal delivery after reaching complete dilatation is a significant marker for true dystocia because it is more likely to recur.

Failure to progress—in either spontaneous or stimulated labor this describes ineffectual labor. Failure to progress is not a diagnosis but an observation.

Inadequate uterine contractions (of less than 180 Montevideo units) were diagnosed in 80% of active phase arrest. However, protraction disorders are less well described, probably because of the time interval required before diagnosing slow progress is undefined. Because of the nature of the problem the decision is postponed. The World Health Organization (WHO) has proposed a labor management “partogram” in which protraction is defined as less than 1 cm/hr cervical dilatation for a minimum of 4 hours. Four hours is a very long time to wait to discover that the labor is not proceeding correctly.

### Over Diagnosis

There are claims that labor progression graphs that included the latent phase and thus appeared flat and portrayed long labors, erroneously influenced early diagnosis of dystocia. The American College of Obstetricians and Gynecologists (ACOG) has suggested that before diagnosis of arrest during first stage of labor is made, both of these criteria should be made:

(1) latent phase has been completed, with CD>=4 cm; and

(2) a uterine contraction pattern of 200 Montevideo units or more for a 10-minutes period has been presented without cervical change.

It is possible that epidural analgesia can slow labor.

Insufficient uterine activity is a common and correctable cause of abnormal labor progress.

During the latent phase the cervix undergoes softening and effacement but only slight dilatation. This phase is characterized by uterine contractions with mild intensity; short duration, and variable frequency. During the active phase the cervix dilates more rapidly and there is descent of the presenting part through the birth canal. The onset of descent is often before the cervix reaches full dilatation and proceeds until the presenting part reaches the perineum. It should be noted that while this pattern is used as a basis for birth monitoring, it is in fact quite variable between patients and for different births for the same patient.

A common treatment error is to diagnose a patient as having protraction (relevant only in an active stage) while, in fact, the patient is in a latent stage.

### Uterine Dysfunction

There are two types of uterine dysfunction:

a. hypotonic uterine dysfunction—where the rise in pressure during contraction is insufficient to dilate the cervix; and

b. hypertonic uterine dysfunction or incoordinate uterine dysfunction—either a basal tone is elevated appreciably or the pressure gradient is distorted, perhaps by contraction of the mid-segment of the uterus with more force than the fundus or by complete asynchronism of the impulses originating in each cornua, or a combination of these two.

### Manual Measurements

Referring back to FIG. 1 in more detail, the following is a list of different manual measurements currently used to help place a patient on the graphs of FIG. 1, in a typical birth process. The information in the parentheses indicate stages where this information is typically obtained.

### Cervix

a) Effacement (mainly in latent phase).

b) Consistency (softness mainly in admittance, latent phase).

c) The presence of membranes with or without amniotic fluid below the presenting part (mainly during admittance).

d) Dilatation (slight in latent+mainly in active stage).

e) Full dilatation (end of active phase).

f) Contractions.

g) Position of the cervix—the relationship of the Cervical os to the fetal head is categorized as posterior, mid-position, or anterior. A posterior position is suggestive of preterm labor.

### Fetal Head

a) Presenting part (admittance).

b) Position=diagnosis of occiput presentation—in 40% of labors the fetus enters the pelvis in left occiput transverse (LOT) position, in 20% is in right occiput transverse (ROT), in 20% the head enters in occiput anterior positions (LOA or ROA), and in 20% it enters in occiput posterior position, where right (ROP) is more common than left (LOP).

c) Station/descent—the degree of descent of the presenting part into the birth canal. Because of the difficulty to reach the pelvic inlet by digital examination, the examiner uses the Ischial spines, which are half way between the pelvic inlet and the pelvic outlet, as a reference point (Mainly in active phase).

d) Engagement—The mechanism by which the bi-parietal diameter (BPD), the greatest transverse diameter of the fetal head in occiput presentations, passes through the pelvic inlet. This may occur during the last weeks before the delivery, or only after the commencement of labor. In multiparas the head is usually not engaged prior to labor. Practically speaking, if the vertex of the head is at 0 station or below, most often engagement of the head has occurred (Mainly in active phase, sometimes in latent phase).

e) Changes in shape of the fetal head—Molding, Caput Succedaneum (Active phase).

f) Asynclitism—The asymmetry of the head transverse axis relative sagittal suture to the birth canal. Moderate asynclitism are considered normal.

g) Flexion—the chin is brought into more intimate contact with the fetal thorax. This usually happens as soon as the descending head meets resistance (Active phase).

h) Internal rotation—turning of the head, is essential for the completion of labor, except for an unusually small fetus. Internal rotation is always associated with descent of the head and is usually not accomplished until the head has reached the level of the spines and therefore is engaged (Active phase).

i) Extension—After internal rotation, the sharply flexed head reaches the perineum and then the vulva, it essentially undergoes extension. (end of active phase, 2nd stage)

j) External rotation (2nd stage).

k) Expulsion (2nd stage).

### Pelvis

Pelvic architecture (admittance)—is reevaluated for adequacy.

### OTHER REFERENCES

U.S. Pat. No. 6,200,279, the disclosure of which is incorporated herein by reference, describes a positioning system for tracking a fetal head.

In a dissertation of Robert Neal Wolfson “An Instrument for the Continuous and Quantitative Determination of Fetal Descent by Measurement of Ultrasonic Transit Time” Department of Biomedical Engineering, Case Western Reserve University, (September 1974), the disclosure of which is incorporated herein by reference, it is suggested to track a fetal head and/or cervix dilatation using ultrasonic position sensors.

U.S. Pat. No. 6,669,653, the disclosure of which is incorporated herein by reference, describes a fetal positioning system, which includes a position-sensor based embodiment and a mechanical device based embodiment using rigid members.

U.S. Pat. No. 5,135,006, the disclosure of which is incorporated herein by reference, describes a ruler, attached to a fetal head and used for monitoring descent.

### SUMMARY OF THE INVENTION

A broad aspect of some embodiments of the present invention relates to a method of monitoring a birth process, in which the birth process is managed as a particularized process in which changes are related to the ongoing process and not merely to a statistical representation of a typical process.

A broad aspect of some embodiments of the invention relates to automatic analysis of collected positional information relating to the position of parts of the cervix and/or fetal head. In an exemplary embodiment of the invention, the analysis includes comparing an input, such as a contraction strength signal, to an output (result) of the contraction, such as motion vector of a fetal head. In an exemplary embodiment of the invention, the analysis makes use of the fact that information is available more accurately and/or more often than by manual measurement, including during contractions.

In an exemplary embodiment of the invention, the particular birth process is divided up into states, each state of which is identified from physiological and/or statistical properties of that birth process, especially, in some embodiments of the invention, geometrical information. In an exemplary embodiment of the invention, the information regarding a state is available in a frequently updated manner, for example, within 10 minutes. In some embodiments and/or situations a faster or slower determination is made, for example, in 5, 2 or fewer minutes, or in 15 or 20 minutes or more. This is in contrast to the prior art, in which a small number of measurements are made and the data is compared to a graph to see a fit. As the graph is an average for many births, even if a high rate of measurements is available, an identification of a state of the birth process will generally not be correct until well after the state has started. Also, even when information is not compared to a graph, deciding on a state often takes an hour or more. It should be noted that in the case of determining whether drugs have an effect, some information may be available relatively fast. Also, in determining fetal distress, a decision is often made within a few minutes, however with a 50% false positive rate.

As used herein the term “state” relates to a step or condition in a birth process which is identifiable. As will be noted below, some states are dynamic in that various parameters change during the state. For example, during an “acceleration stage” state, cervical dilatation increases. A static state, for example a “fetal engagement” state, generally indicates a clearly identifiable milestone in the birth process. As can be appreciated, it may be desirable, and is possible in some embodiments of the invention, to identify such static and/or dynamic states in a short time.

As an example of a dynamic state, a maximum slope state is a continuum and comprises a continuous range of dilatation values and a certain pattern is expected to be imposed on these values (e.g., a linear increase).

In some embodiments a state may be a sub-state of another state. In some embodiments, states may occur in parallel. In particular, the state of the fetal head can progress along one time path, while the state of the cervix progresses along another time path. In some cases, overlap of states is normal. In others cases, an overlap of states is used to identify an abnormal labor state, for example based on the overlapping states not being typically co-occurring.

Optionally, the determined state is shown on a standardized curve. However, in an exemplary embodiment of the invention, the state is shown on a state based display in which the various physiological parameters are shown as expected or actual values for the current birth, rather than, or in addition to, average values for many births. Optionally, a subset of births is selected to be used as a statistically calculated background to the present display, for example, selecting births with similar parameters or progress.

It should be noted that the particular methods of determining a state and/or state information may be practiced also as part of other birth monitoring methods.

In an exemplary embodiment of the invention, the state is identified based on relative positions of sensors during contractions. In other embodiments, relative positions of the sensors during contraction are used, instead of in addition to differences in position between contractions and the non-contraction intervals.

In an exemplary embodiment of the invention, motion during a contraction is used as a predictor of motion over time. For example, a vector of fetal head motion during a contraction is used to predict the direction of motion of the head after a sufficient number of contractions (and associated progress) has occurred. Optionally, the degree of motion of a fetal head for a certain cervical intra-contraction dilatation is used as an indicator for rate of expected progress and estimated progression time to a later state. Mismatch of actual progress to an expected progress determined in this way, may be an indication of abnormal progress.

In accordance with an exemplary embodiment of the invention, a birth management system is provided which includes one or more sensors attached to a patient and which generates an indication of a state of a birth process. Optionally, the system provides a prognosis, for example, expected future states. Optionally, the system assists in diagnosis, for example providing a diagnosis of a problematic situation, such as “Arrest Disorder”. Optionally, the system indicates an expected time to complete a current or future state. Optionally, the system indicates the progress of labor as compared to an expected progress. In an exemplary embodiment of the invention, the system stores (e.g., in a database) a plurality of exemplary birth processes, for example for the same patient, other patients and/or manually entered data. Optionally, the system compares the current birth process to this database, to find a best match, for estimating future time and/or to anticipate expected problems. Optionally, the system includes means for monitoring various accepted treatments protocols. For example, if an attendant indicates that a certain drug treatment is to be used, the system will display a series of states and/or decision points which an attendant and patient are expected to go through. In an exemplary embodiment of the invention, prognosis is based on one or more of duty factor (percentage of contraction cycle a contraction is in force), area under graph and frequency of contractions.

Optionally, alternatively or additionally to using sensors to directly detect states, statistically defined states are identified based on statistical analysis. For example, an “acceleration phase” state during a first stage of labor may be identified based on changes in rate of cervical dilatation. In one implementation, the onset of an acceleration stage is detected by collecting dilatation measurements over a plurality of contractions, for example, over a period of 10 minutes and statistically processing the results to see if dilatation rate changed significantly. In some situations and/or embodiments, a significant change is one of 10%, 20%, 30%, or any smaller, intermediate or larger number. This is in contrast to prior art methods in which a relatively small number of measurements are generally made and they are compared to the graph of FIG. 1. It is noted that even such a statistical state is generally determined in a short period of time, for example shorter than 20 minutes or shorter than 15 minutes.

In an exemplary embodiment of the invention, the exiting of the fetus from the uterus is tracked, for example by detecting patterns of cervical dilatation and contraction corresponding to, for example, passage of one or both shoulders.

In an exemplary embodiment of the invention, instead of using sensors, markers or transmitters are used. Optionally, an imaging system is used (e.g., instead of transponders), optionally including software to automatically detect features of the cervix and/or fetal head. Alternatively, other position sensing methods known in the art can be used, for example US (ultrasound), RF and DC magnetic field based methods.

An aspect of some embodiments of the invention relates to using a head station vector to monitor a birth process. In an exemplary embodiment of the invention, the direction of the vector is compared to an expected birth path and used to estimate whether the labor is progressing. Optionally, the vector is used to detect one or more of flexing and engagement. Optionally, a combination of cervical dilatation variation and head station vector is used to detect a failure to progress situation.

An aspect of some embodiments of the invention relates to using the state of labor to help decide what treatment to give and/or to control such treatment. For example, if the system detects that the first stage of labor is not completed yet, it will prevent (or suggest to prevent) the administration of certain drugs which might otherwise lead to fetal distress. Optionally, the system will increase or decrease drug dosages (e.g., using a semi-automatic or fully automatic feedback loop), or change an administered drug depending on whether the current drug and dosage are effective.

An aspect of some embodiments of the invention relates to using a detection of a state of labor to provide corrected input. For example, once full dilatation is reached, the system may display a dilatation of 10 cm, even if the real dilatation is only 9.0 cm. Optionally, also intermediate values of cervical dilatation are “corrected” to yield values of the type currently reported by a physician, for example, if about 1 cm remains to full dilatation, a value of 9 cm will be reported regardless of the real dilatation, or beside it. Optionally, the system maintains the continuity of the “corrected” value. Optionally, at full dilatation, the system displays a notice that the actual dilatation is not “10 cm” but actually some other number, which is optionally displayed. It should be noted that while the cervical dilatation is modified, the actual displayed value may be more correct only in that it fits accepted scales and not with respect to geometrical correctness.

In an exemplary embodiment of the invention, a display of dilatation value, even prior to full dilatation is corrected from a measured value to match numbers that are used as a nomenclature. Optionally, this correction causes real measurements to be changed into values which would probably (e.g., on an average) be reported by a human using digital manipulation, even though not numerically correct.

Optionally, the values of cervical dilatation and/or head station may take into account their variability due to contractions to correct their values, for example by adding a certain factor to dilatation in the presence of significant changes during contractions. It is expected that this correction may lead to values closer to those reported by humans using digital manipulation, e.g., if there is a large variability, humans will tend to overestimate because the cervix is more flexible. Optionally, the correction assumes that the effect of contraction and the effect of digital pressure on the cervix is similar (e.g., for a same magnitude). Optionally, the force applied by a doctor is measured (optionally storing values for different doctors). An estimate of elasticity of the cervix is optionally obtained by comparing CD (cervical dilatation) variability with contraction strength. This estimate of elasticity is optionally used to generate a correction from the CD measured to the value that it is expected a human would report for that state.

In an exemplary embodiment of the invention, new birth states are defined that are not currently recognized. In one example, once the Cervical os starts retracting relative to the fetal head a state of “approaching full dilatation” is defined, which indicates that full dilatation is imminently expected. Such new states may be presented to a user or be internal to the system. In another example, a “Head to cervix” state is defined when the fetal head is working against the cervix. These dynamics are optionally represented in one or both of qualitatively (existence) and quantitatively (strength). In another example, an “Effective contraction” state is defined, indicating that uterine contractions are effective, optionally reflecting increase in amplitude and in net progress (δ) of descent and dilatation.

An aspect of some embodiments of the invention relates to detecting an onset of a second stage of labor. In an exemplary embodiment of the invention, the second stage of labor, or at least full dilatation, is determined to start when a Cervical os passes a BPD of a fetal head. In an exemplary embodiment of the invention, this passage is detected by measured relative positions of the Cervical os and the fetal head, for example using a medical imager or using one or more relative or absolute position sensors. Alternatively or additionally, this passage is determined by movement of the Cervical os and/or the fetal head relative to an anatomic feature of a mother, for example an ISL (Ischial spines) or an ASIS (Anterior superior iliac spines). Optionally, the second stage is detected based on a feature of the contraction pattern, for example, a great reduction in variability of CD, not accompanied by reduction in HS variability.

In an exemplary embodiment of the invention, a full dilatation (i.e., end of stage I) is determined by a cresting of a Cervical os over a fetal head. While standard progression figures of a birth process apparently show the correct position of a Cervical os, it has apparently not been heretofore realized, that full dilatation can be characterized and identified by the Cervical os passing from one side of a fetal head maximum diameter (also known as BPD—bi-parietal diameter) to an opposite side thereof. This crestal transit may involve a sudden change in relative position of the fetal head and the Cervix and/or a sudden retrograde motion of the Cervical os, possibly not as part of a contraction, from a state in which the fetal head pushes the Cervical os forward to a state in which the Cervical os slips past part of the head. In an exemplary embodiment of the invention, the crestal transit is detected by comparing a level of the sensor(s) of the Cervical os to a reference point, for example on the fetal head and/or on the paternal body.

An aspect of some embodiments of the invention relates to a method of determining a relative descent of a fetal head sensor and one or more cervical sensors, taking into account the possible three dimensional configurations of the fetal head and cervix. In an exemplary embodiment of the invention, a projection of the descent is provided on a path of motion of the fetal head. In an exemplary embodiment of the invention, a relatively distant (compared to distance between sensors) reference point or line is provided for comparing the sensor positions to, for example one or both ASIS (anterior superior iliac spine) being used as a reference point. The distances of the cervical and head sensors to this reference point (or line) are compared to determine head descent. In one example, a distance between the line and a head sensor is compared to an average distance between cervical sensors and the line. Optionally, the line is determined using position sensors attached outside the body.

An aspect of some embodiments of the invention relates to using statistical information about head station (HS) and/or cervical dilatation (CD) to determine a birth state and/or detect abnormal states. In an exemplary embodiment of the invention, the variations (changes) in a measure such as CD or HS during a contraction are used. Several different parameters based on the variations may be defined, for example, maximum variation, variation changes over time (variability) and normalized variation or variability. In an exemplary embodiment of the invention, the variability of the cervical dilatation and optionally of the head station is used to distinguish between two or more of a latent phase (low CD variability), an acceleration phase (medium CD variability), a maximum slope stage (high CD variability) and a deceleration stage (low CD variability, but high HS variability). Optionally, this information is correlated with head station, which is expected to increase linearly starting at the very end of the latent phase. Optionally this information is correlated with input from a fetal monitor, in particular fetal heart rate and/or TOCO/IUP. Various pathological states can be identified from deviations from this progression and/or a mismatch between dilatation variation and head station (e.g., low head station value, high variability). Non-pathological states may also be identified. For example, the correlation descent-dilatation variability can be used to identify and quantify the “Head to Cervix” state.

In a particular exemplary embodiment of the invention, cervical dilatation variation is used to decide if a labor is arrested or if is still in a latent phase.

In some embodiments of the invention, monitoring is based completely or in large measure (e.g., at least for some types of important information) on variation information. This may allow to use fewer sensors and/or avoid a baseline calibration stage.

An aspect of some embodiments of the invention relates to determining a functionally effective fetal head station. In an exemplary embodiment of the invention, a functionally effective head station is determined based on changes in orientation of the head relative to the rest of a fetal body and/or a mother, rather than solely on positional advance along a birth canal. In an exemplary embodiment of the invention, one or more of “cervix”, “engagement”, “flexion”, “at bend in canal”, “internal rotation”, “perineum”, and “extension” are used to define a functionally effective fetal head station, instead of distance measurements. Optionally, the distance measurements are translated into states, for example “at a bend in the birth canal”. Optionally, a patient anatomical measurement is made, for example using imaging methods known in the art so that the station numbers can be provided as normalized numbers.

In an exemplary embodiment of the invention, one or more of the above geometrically-related values are used to distinguish between various labor patterns associated with various positions of fetal head. Each such geometry may have, for example, a different prognosis, a different set of potential problems (or problem probabilities) and/or a different set of expected states to go through. A particular expected pattern may also be selected and/or fine-tuned using pelvic anatomy and motion and/or orientation of maternal and fetal sensors in three dimensions.

In an exemplary embodiment of the invention, a ‘Pelvic division’ state, which corresponds to a birth canal station, is identified by a change in fetal head orientation. In an exemplary embodiment of the invention, the division is detected by determining internal rotation followed by extension, which is caused by a bend in the birth canal that requires the fetal head to rotate and the neck to bend. In an exemplary embodiment of the invention, the fetal head orientation is detected using one or more sensors on the fetal head. Alternatively or additionally, the orientation is detected based on an effect of the head rotating and neck bending on the Cervical os, for example, changing its plane of opening.

An aspect of some embodiments of the invention relates to determining the effectiveness and/or type of uterine contractions. Such determining optionally includes noise reduction and/or other filtering. In an exemplary embodiment of the invention, the determined effectiveness and/or variations thereof is used to determine the state of labor (e.g. latent phase, active phase, maximum slope). In an exemplary embodiment of the invention, the determined effectiveness and/or variations thereof are used to determine pathologies such as baby arrest, protraction, and failure to progress. Optionally, a ‘contraction activity’ measure is determined, which comprises the change of dilatation due to contraction. Optionally, this measure is used to calculate mechanical parameters such as the force exerted on the fetal head (e.g., pressure times surface area or motion in response to pressure) and/or tissue compliance. An optional measure determined is the net progress (δ) due to individual contraction, i.e. the CD post contraction minus CD before contraction. Optionally, the relationship between two or more of CD variability, HS variability and CD net progress is used for diagnosis and/or state identification. Optionally, the measure of intra uterine pressure (IUP) is used in conjunction with the above for diagnosis and/or state identification.

In an exemplary embodiment of the invention, the form of the contractions is used as a criterion for effectiveness. The present inventor has noted that a typical measurement system, such as TOCO cuts off low amplitude parts of a contraction, thus creating the impression that a typical contraction erupts from a plateau of non-contraction. In contrast, measurements of the Cervical os in accordance with exemplary embodiments of the invention show substantially continuous (e.g., an undulating line) variation during effective contractions. Such a continuous line often represents a duty factor of up to 50% (and possibly more). In an exemplary embodiment of the invention, a high duty factor, optionally in conjunction with sufficient amplitude and/or frequency, indicates that the contractions are effective. For example, if the duty factor is low, a medication such as Pitocin, may be prescribed (or a pre-labor state identified).

In an exemplary embodiment of the invention, a contraction signal is filtered using physiological considerations. In an exemplary embodiment of the invention, limits on the rate of increase or decline of contraction amplitude is used as a filter. Any potential contraction which has rise and/or decline rates above a threshold may be identified as noise (e.g., motion artifact) and removed. In an exemplary embodiment of the invention, the filtration is by differentiating, filtering and then integrating a signal. Optionally, a baseline is not restored during integration.

In an exemplary embodiment of the invention, a state of labor is detected from the shape of contractions. In one example, a first, higher, ratio between rise rate and decline rate is used to characterize the active phase and a second, lower, ratio between rise rate and decline rate is used to characterize the pelvic stage. This is true at least for some women. In observation it was found that during an active phase the HS rise rate is between 1-4 mm/sec and typical decline rates is between (0.5)-(2.5) mm/s, whereas, normal expulsion contractions have a HS change magnitude of at least 1.5 cm, with a typical rise rate of 5-20 mm/sec, and a typical decline rate of (3)-(15) mm/sec. As can be seen, both the ratios and the actual values change between states. In an exemplary embodiment of the invention, the asymmetry is detected and the actual interpretation may depend on other birth parameters.

In an exemplary embodiment of the invention, the type of contraction and/or its onset and/or its ending (or some point relative to start or end or other feature thereof) are used for coaching the mother to push during expulsion contractions. In an exemplary embodiment of the invention, a mother is not coached to push or is coached not to push if an expulsion contraction is not detected (or a non-effective contraction is detected) or when a contraction is completed. Optionally, the monitoring system includes a speaker to generate tone or speech instructions and/or encouragement to the mother. Optionally, the monitoring system detects the effectiveness of the maternal actions, for example, detecting motion of the head correlated to contraction strength and maternal action (e.g., as measured by abdominal straining). The head motion may be normalized to typical motion per contraction at different parts of the birth canal, weights, etc. The detected effectiveness may be used to generate new instructions to the mother. Optionally, the monitoring system uses one or more inclination sensors on the mother to coach the mother into changing position (e.g., roll and/or pitch). In an exemplary embodiment of the invention, the monitoring system measures instinctive (or spontaneous) actions by the mother and generates an indication, for example, if such actions are normal, effective and/or abnormal. Examples of such instinctive behavior is bending forward during a contraction, abdominal pressure increase during a contraction and/or shallow breathing and/or other change sin breathing.

Optionally, the monitoring system is used with a walking epidural, for example, to instruct a patient to carry out certain birth-related exercises or to request stopping motion so that a more accurate measurement may be made.

In an exemplary embodiment of the invention, the shape of the contractions and/or their duty factor and/or amplitude are used to identify abnormal contracts. The above noted ratios between rise and decline may characterize one or more of pressure, CD and/or HS.

An aspect of some embodiments of the invention relates to control of a labor process. In an exemplary embodiment of the invention, inputs, for example, drugs or body position are varied in a manner suitable to improve an effectiveness of contraction and thus speed up labor and/or make it more efficient. In an exemplary embodiment of the invention, these inputs are varied to improve one or more of the safety of the fetus and/or mother and/or the speed of delivery. For example, once uterine contractions are as effective as can be (e.g., as measured by geometry result, not pressure) there is no need to make them stronger.

In an exemplary embodiment of the invention, the effectiveness of uterine contractions is used to regulate the titration of drugs such as Oxytocin and Pitocin (or their antagonists), so that such drugs are not used in an incorrect stage of labor. Optionally, the titration of drugs is fine tuned to a better temporal resolution, for example, being varied on a contraction-to contraction basis or every small number of contractions (e.g., 2-3), as needed. The regulation may be time based, for example, changing titration every 10 or 20 minutes, as needed.

An aspect of some embodiments of the invention relates to displaying of information, for example, integrating physiological sensor information (non-geometric, such as pressure or heart rate) and geometrical information to provide meaningful results. In one example, a TOCO gauge or an IUP sensor are used to indicate the existence of a contraction, while geometrical information is used to assess its effect. In another example, geometrical information is used to calibrate or indicate a measurement problem in other sensors. In another example, the synchronization of events can be determined, for example, synchronization of fetal bradycardia with cervical dilatation. This may assist in indicating the cause of the bradycardia and/or suitable treatment.

Optionally, the integration allows a more in depth analysis. For example, the pressure on a fetal head can be determined using a physiological model which takes into account pressure on one hand and compliance of the cervix on the other.

Optionally, physiological information includes one or more of maternal information, and fetal information, including, for example, vital signs and pressure measurements of the contractions, internal and/or external.

In an exemplary embodiment of the invention, the display includes interventional information, such as provision of medication, manual examination and instructions to the mother, for example, position changes. In an exemplary embodiment of the invention, the birth monitoring system is used to track compliance of the mother with such instructions for example, changes in posture, breathing rate and/or pushing. Optionally, the display includes an indication of the effectiveness of the intervention, for example, a desired effect (e.g., increase in contraction amplitude or duty factor) and the actual effect (e.g., using a numerical definition, such as % increase in duty factor).

In an exemplary embodiment of the invention, such displaying and/or integration comprises comparing the input (e.g., contraction pressure or electrical signals) with an output, (e.g., the geometrical effect of a contraction). Also envisioned is determining the effect of a contraction on a fetal heart rate.

In an exemplary embodiment of the invention, the displaying using statistical analysis is a concise manner of showing a current status of birth as compared to the general process. For example, showing a histogram of variation values as a function of cervical dilatation allows an easy determination if current measurements fit an existing pattern of the patient. In an exemplary embodiment of the invention, the use of a concise and/or statistical presentation allows the data to be normalized. In the example of the histogram, the shape of the distribution will be the same even if in a patient the cervical dilatation measurements are all off by a certain value or if the variation values in general for that patient are higher.

An aspect of some embodiments of the invention relates to determining two or more of a magnitude of a contraction, an efficacy of a contraction and/or an efficiency of a contraction (or a set of contractions). Optionally, one or more of these measures are obtained from geometrical information, possibly in conjunction with physiological information.

An aspect of some embodiments of the invention relates to detection of head inflation, in which a fetal head expands due to accumulation of fluid between the skull and the scalp at the apex of the head. Such inflation may cause a sensor attached to the skull to indicate fetal head movement, when, in fact, there may be no movement or a different movement. In an exemplary embodiment of the invention, such inflation is detected directly by a sensor attached to the fetal head and which monitors, for example using ultrasound, the distance to the skull. In an exemplary embodiment of the invention, such inflation is detected indirectly by determining apparent head descent while no cervical dilatation is present.

An aspect of some embodiments of the invention relates to apparatus for detecting the onset of a second stage of labor. In an exemplary embodiment of the invention, a device is mechanically coupled to a Cervical os and when that Cervical os retracts significantly relative to a reference point inside or outside the body, an indication is generated. In an exemplary embodiment of the invention, the device comprises a cervical anchor attached to a ruler, which ruler is long enough to exit the vagina. When the Cervical os retracts, the length of the ruler outside the body shortens. Optionally, an alarm is attached to the ruler, for example, the device including an element attached to the outside of the body, so that retraction of the ruler relative to the attachment element generates an audible and/or radio alarm. Such an alarm may be used to replace frequent (or too infrequent) manual checking of a patient's state.

An aspect of some embodiments of the invention relates to estimating effacement. In an exemplary embodiment of the invention, an estimation of effacement is obtained, at least during a state where there is a small cervical dilatation (<3 cm, for example), by determining the relative head descent between a fetal head sensor and a cervical sensor(s), optionally using a remote reference point or line. Optionally, the estimate includes a correction for estimated flexibility (e.g., tissue consistency). Such an estimate of flexibility may be obtained for example, by measuring the stretching of the Cervical os when manual measurement is made or extracting a relationship between CD variability and strength of contraction.

There is thus provided in accordance with an exemplary embodiment of the invention, a method of monitoring a birth process, comprising:

receiving, over time, a plurality of position signals from one or more positioning elements or tissue areas located at least one of a cervix and a fetal head; and

determining a discrete state of labor of a fetus that is wholly inside a body responsive to said position signals, with a temporal resolution of better than 15 minutes, said discrete state being other than a start or stop of labor and encompassing more than a single contraction, said state including a state other than an abnormal fetal head position.

Optionally, said one or more positioning elements comprises a wireless transponder. Alternatively or additionally, receiving comprises receiving from one or more tissue areas identifiable using an imaging system.

Alternatively or additionally, receiving comprises receiving from at least one positioning element.

Alternatively or additionally, said one or more positioning elements comprises a transmitter.

Alternatively or additionally, said one or more positioning elements comprises a marker.

In an exemplary embodiment of the invention, said discrete state comprises at least one state from a list of states including: failure to progress, inefficient uterine contractions, onset of active labor, full dilatation, optimal uterine activity, individual maximum slope of dilatation, fetal head internal rotation, fetal head extension, pre-cresting, arrest disorder, canal arrest, abnormal expulsion contractions, normal expulsion contractions, efficacy of drug administration and readiness for delivery. Optionally, the method comprises determining at least 2 states from said list at different times. Optionally, the method comprises determining at least 4 states from said list at different times. Optionally, determining at least 6 states from said list at different times.

In an exemplary embodiment of the invention, the position signals comprises fetal head position signals and cervical OS position signals.

In an exemplary embodiment of the invention, the position signals do not comprise absolute cervical dilatation signals.

In an exemplary embodiment of the invention, the position signals comprise absolute cervical dilatation signals.

In an exemplary embodiment of the invention, the method comprises modifying the cervical dilatation signals to reflect a scale on which full dilatation is 10 cm.

In an exemplary embodiment of the invention, determining comprises determining based on an analysis of short term changes in said signals, within a time period of a contraction cycle. Optionally, said analysis comprises an analysis of changes in a fetal head position. Optionally, said analysis comprises an analysis of a spatial vector of fetal head motion.

In an exemplary embodiment of the invention, said analysis comprises an analysis of changes in cervical geometry.

In an exemplary embodiment of the invention, said analysis comprises an analysis of rate of change of a position.

In an exemplary embodiment of the invention, said analysis comprises an analysis over a plurality of contractions.

In an exemplary embodiment of the invention, said determining comprises determining based on a duty factor of a plurality of contractions.

In an exemplary embodiment of the invention, said determining comprises determining that a labor is progressing normally.

In an exemplary embodiment of the invention, said determining comprises determining that a labor is progressing abnormally.

In an exemplary embodiment of the invention, said determining comprises determining a type of contraction.

In an exemplary embodiment of the invention, said determining is based on non-geometrical physiological signals of at least one of mother and fetus. Optionally, said determining comprises analyzing a phase delay between non-geometric physiological and geometrical measurements. Alternatively or additionally, said physiological signals comprise pressure signals. Alternatively or additionally, said physiological signals comprise EMG signals. Alternatively or additionally, said physiological signals comprise heart rate signals.

In an exemplary embodiment of the invention, determining comprises determining a state on a personalized time/progression scale.

In an exemplary embodiment of the invention, the method comprises matching a progression of labor to one of a plurality of templates.

In an exemplary embodiment of the invention, the method comprises estimating a time to reach a future state, based on said signals.

In an exemplary embodiment of the invention, said position signals are acquired using a reference remote from said elements.

In an exemplary embodiment of the invention, the method comprises determining at least one of an orientation change and magnitude change in a vector of a fetal head. Optionally, said change in vector comprises a change in orientation of a fetal head. Alternatively or additionally, the method comprises generating a head station value indicating the spatial progression of the fetal head in a birth canal. Alternatively or additionally, said vector comprises a vector of motion of said head during a contraction. Alternatively or additionally, the method comprises comparing said vector to an expected head path in a maternal body. Alternatively or additionally, the method comprises determining an asymmetry between forward motion and backward motion of said head.

There is also provided in accordance with an exemplary embodiment of the invention, a method of labor management, comprising:

(a) collecting information about a labor process;

(b) generating a personalized progression representation based on said information;

(c) identifying a relationship between a parameter of said representation and a norm, within 20 minutes of said parameter changing its relationship relative to the norm; and

(d) selectively modifying a treatment of the labor responsive to said identification. Optionally, said identifying comprises identifying by computer circuitry.

In an exemplary embodiment of the invention, the method comprises suggesting a modification by computer circuitry.

In an exemplary embodiment of the invention, identifying comprises identifying that said parameter is outside a norm.

In an exemplary embodiment of the invention, identifying comprises identifying that said parameter is inside a norm.

In an exemplary embodiment of the invention, selectively modifying comprises not modifying.

In an exemplary embodiment of the invention, generating said personalized progression representation comprises statistical analysis of said collected information. Optionally, said statistical analysis comprises long term analysis. Alternatively or additionally, said statistical analysis comprises short-term analysis. Alternatively or additionally, said statistical analysis comprises generating a histogram.

In an exemplary embodiment of the invention, said personalized progression representation includes an expected rate of change.

In an exemplary embodiment of the invention, said personalized progression representation includes an identification of at least three labor states.

In an exemplary embodiment of the invention, said personalized progression representation comprises an indication that an individual maximum slope is about to be achieved. Optionally, said indication comprises a dedicated display.

In an exemplary embodiment of the invention, said indication comprises a state display including a presentation of states according to their relative context and including a history of states.

In an exemplary embodiment of the invention, said indication comprises a display of individual maximum slope.

There is also provided in accordance with an exemplary embodiment of the invention, a method of monitoring a labor process, comprising:

receiving, over time, a plurality of positional information from one or more positioning elements or tissue segments located at least one of a cervix and a fetal head;

determining at least one change in magnitude of positional information within a contraction;

analyzing said at least one change; and

determining a status of said labor based on said analysis.

Optionally, the method comprises analyzing over a plurality of contractions to yield a composite indication used in said determining.

In an exemplary embodiment of the invention, said analysis comprises maximum change analysis.

In an exemplary embodiment of the invention, said analysis comprises rate of change analysis.

In an exemplary embodiment of the invention, said analysis comprises analysis of cervical dilatation.

In an exemplary embodiment of the invention, said analysis comprises analysis of fetal head position.

In an exemplary embodiment of the invention, said analysis comprises analysis of a duty factor of the contraction based on changes in position.

In an exemplary embodiment of the invention, determining a state comprises determining a discrete state.

In an exemplary embodiment of the invention, the method comprises displaying said analysis in a graphical form. Optionally, said graphical form shows results for at least two hours of said labor. Alternatively or additionally, said graphical form shows results for at least half an hour of said labor. Alternatively or additionally, said graphical form shows results for at least 10 contractions. Alternatively or additionally, said graphical form shows results for at least 30 contractions.

In an exemplary embodiment of the invention, determining comprises determining based on non-geometric physiological information.

In an exemplary embodiment of the invention, determining comprises determining based on long term net progression between contractions.

In an exemplary embodiment of the invention, the method comprises generating an indication of an effectiveness of said contraction. Optionally, the method comprises generating an indication of an effectiveness of a drug titrated in said labor. Alternatively or additionally, the method comprises generating an instruction to a mother regarding pushing based on said indication.

In an exemplary embodiment of the invention, the method comprises normalizing said change based on measurements from a current labor.

In an exemplary embodiment of the invention, the method comprises normalizing said change based on a currently identified state of said labor.

There is also provided in accordance with an exemplary embodiment of the invention, a method of reporting a cervical condition, comprising:

measuring a cervical dilatation; and

modifying said measurement other than by sensor calibration to generate a different dilatation value smaller than or equal to 10 cm. Optionally, said modifying comprises correcting said measurement to reflect a human nomenclature where 10 cm indicates full dilatation. Alternatively or additionally, said modifying is applied only for measurements larger than 5 cm. Alternatively or additionally, said modifying is applied based on a detection of fetal head cresting. Alternatively or additionally, said correction comprises a correction for the compliance of the cervix. Alternatively or additionally, said correction is personalized to correct for a bias of a practitioner making the measurements. Alternatively or additionally, said correction is personalized per patient.

There is also provided in accordance with an exemplary embodiment of the invention, a method of detecting full dilatation of a cervix, comprising:

measuring a relative position of a cervix and a reference point; and

determining full dilatation when said cervix moves relative to the reference point in accordance with a predetermined motion. Optionally, the reference point comprises a fetal head. Alternatively or additionally, determining comprises detecting that said cervix crests over said fetal head. Alternatively or additionally, said relative positions are determined relative to a virtual point in space, distanced from said head and cervix and in a direction of motion of the fetal head.

In an exemplary embodiment of the invention, the suitable manner comprises retrograde motion of said cervix.

There is also provided in accordance with an exemplary embodiment of the invention, a method of determining a relative position of a point on a fetal head and a point on a cervix, comprising:

determining distances of the points from a reference location distanced from the sensors and in a general direction of an expected motion of said fetal head; and

determining relative values of the distances. Optionally, the method comprises determining effacement of a cervix based on motion relative to said reference point. Alternatively or additionally, the method comprises detecting cresting of said fetal head based on motion relative to said reference point. Alternatively or additionally, the method comprises not reconstructing a plane of an opening of said cervix os.

There is also provided in accordance with an exemplary embodiment of the invention, a method of monitoring a labor process, comprising:

collecting geometrical information about an effect of a contraction;

collecting non-geometric physiological information about an effect of a contraction; and

correlating the collected geometric and non-geometric information. Optionally, comprises displaying in a same time line. Optionally, the method comprises displaying labor events in the same time line. Alternatively or additionally, correlating comprises determining a phase difference between the non-geometric and geometrical information.

In an exemplary embodiment of the invention, said geometric information comprises changes in geometrical information within a contraction cycle.

In an exemplary embodiment of the invention, said geometric information comprises cervical dilatation and fetal head position. Optionally, the method comprises presenting one of geometric information and non-geometric information as a function of the other. Optionally, the method comprises presenting the informations in histogram form.

In an exemplary embodiment of the invention, the method comprises gating one of geometric information and non-geometric information as a function of the other.

In an exemplary embodiment of the invention, the method comprises presenting the informations in strip form.

In an exemplary embodiment of the invention, the method comprises presenting the informations as an overlay of information from different contractions.

In an exemplary embodiment of the invention, the method comprises presenting the informations in three-dimensional form.

There is also provided in accordance with an exemplary embodiment of the invention, a method of detecting a potential fetal head deformation, comprising:

detecting a putative head descent condition;

detecting a cervical dilatation value;

determining a mismatch between the head descent and the cervical dilatation value; and

determining a deformation based on said mismatch. Optionally, said cervical dilatation value is a less than full dilatation. Alternatively or additionally, said cervical dilatation is determined to be a pre-cresting state. Alternatively or additionally, said detecting a condition and said detecting a value comprise detecting using an attached positioning element.

There is also provided in accordance with an exemplary embodiment of the invention, apparatus for detecting an onset of second stage of labor by cervical retrograde motion, comprising:

(a) an engager adapted to engage a Cervical os; and

(b) a body coupled to said engager and adapted to show a retraction of said engager relative to a body of a patient. Optionally, said body is elongate enough to extend outside of a patient when attached to cervix os. Alternatively or additionally, the apparatus comprises an audible alarm activated upon detection of said retraction.

In an exemplary embodiment of the invention, said body includes a ruler.

In an exemplary embodiment of the invention, said ruler is adapted for calibration of initial position of said cervix.

In an exemplary embodiment of the invention, the apparatus comprises the method comprises a mark of an initial position of said body.

There is also provided in accordance with an exemplary embodiment of the invention, a method of estimating changes in a cervical os, comprising:

(a) collecting positional information from at least one of a positioning element located on a fetal head and a positioning element located on the cervical os; and

(b) analyzing the positional information to yield an estimate of a cervical os property other than dilatation. Optionally, said analyzing comprises estimating an effacement from a degree of fetal head motion. Alternatively or additionally, said analyzing comprises estimating a resiliency by comparing a change in cervical dilatation to a strength of a contraction. Optionally, said strength is measured using an IUP (intra-uterine pressure) sensor.

In an exemplary embodiment of the invention, said analyzing comprises comparing a machine measurement of cervical dilatation to a human estimate of cervical dilatation.

In an exemplary embodiment of the invention, said analyzing comprises determining rotation of a cervical positional element.

In an exemplary embodiment of the invention, said collecting comprises collecting during an intervention. Optionally, said intervention comprises a manual examination.

There is also provided in accordance with an exemplary embodiment of the invention, a method of filtering geometrical labor information, comprising:

(a) providing a stream of geometrical information from a labor process; and

(b) filtering the stream using a filter that rejects data that is physiologically incorrect. Optionally, the method comprises rejecting data based on a length of contraction. Alternatively or additionally, said filter rejects data based on their derivative. Optionally, filtering comprises:

finding a derivative for said data;

thresholding the data; and

integrating the data.

There is also provided in accordance with an exemplary embodiment of the invention, a method of controlling of pharmaceutical provision to a patient in labor, comprising:

(a) providing an intervention to the patient;

(b) collecting information on geometrical changes in said patient indicating an effect of the intervention on a labor process; and

(c) selectively modifying said providing in response to said collecting with a feedback time of less than 20 minutes. Optionally, said feedback time is less than 10 minutes. Alternatively or additionally, the method comprises maintaining a desired range of geometrical response by said modifying.

In an exemplary embodiment of the invention, said modifying comprising stopping said providing if no labor progression is generated by said intervention

In an exemplary embodiment of the invention, said modifying comprising modifying said intervention to achieve a maximal individual slope for the patient.

In an exemplary embodiment of the invention, said intervention comprises pharmaceutical provision.

In an exemplary embodiment of the invention, said intervention comprises an instruction to change position.

In an exemplary embodiment of the invention, said selectively modifying comprises automatically selectively modifying.

In an exemplary embodiment of the invention, said selectively modifying comprises generating a suggestion to selectively modify.

There is also provided in accordance with an exemplary embodiment of the invention, apparatus for monitoring labor, comprising:

(a) an input adapted to receive input signals from at least one monitoring system monitoring a patient in labor; and

(b) a controller configured to carry out any of the preceding methods based on the received signals. Optionally, the apparatus comprises an instruction output which displays instructions to a patient in labor. Optionally, the apparatus comprises a tracker adapted to track the effect of such instruction on said signals.

Optionally, the apparatus comprises a monitor adapted to monitor compliance with said instructions.

There is also provided in accordance with an exemplary embodiment of the invention, a method of presenting geometrical information collected during a labor process, comprising:

(a) arranging positional information from at least one cervical position and at least one fetal position in a 3D display; and

(b) arranging the display to maintain a center of gravity between positions of said sensors. Optionally, the method comprises arranging state information on said display. Alternatively or additionally, the method comprises arranging variability information on said display.

### BRIEF DESCRIPTION OF THE FIGURES

Non-limiting embodiments of the invention will be described with reference to the following description of exemplary embodiments, in conjunction with the figures. The figures are generally not shown to scale and any sizes are only meant to be exemplary and not necessarily limiting. In the figures, identical structures, elements or parts that appear in more than one figure are preferably labeled with a same or similar number in all the figures in which they appear, in which:

FIG. 1 is a graph showing cervical dilatation as related to birth progression, in accordance with what is known in the art;

FIG. 2 (split into FIGS. 2A and 2B, for pagination reasons) is a tree-type state diagram showing various states of a birth process, at least some of which are identified in accordance with an exemplary embodiment of the invention;

FIGS. 3A-3B are schematic illustrations showing the relative position of a Cervical os and a fetal head, during dilatation;

FIG. 3C is schematic illustration showing cresting of the fetal head relative to the Cervical os, at full dilatation;

FIGS. 3D and 3E are side cross-sectionals view of a birth process, showing cresting;

FIG. 3F is a cross-sectional anatomical view showing various landmarks useful for carrying out some exemplary embodiments of the invention;

FIG. 3G is a schematic illustration showing the extraction of relative cervical and fetal head positions using a remote reference in the form of a line connecting the ASIS, in accordance with an exemplary embodiment of the invention;

FIG. 4A schematically shows a correspondence between cervical dilatation variation and head station variation, as used in accordance with an exemplary embodiment of the invention;

FIG. 4B shows a correspondence between cervical dilatation and head station, which can be used in accordance with an exemplary embodiment of the invention;

FIG. 4C shows synchronized traces for geometrical and physiological sensors, in accordance with an exemplary embodiment of the invention;

FIG. 4D is a flowchart of a method of modifying cervical dilatation measurements, in accordance with an exemplary embodiment of the invention;

FIGS. 5A-5D illustrate a normal mechanism of labor for a left occiput transverse position, in lateral view, which can be monitored in accordance with an exemplary embodiment of the invention;

FIG. 6 illustrates a normal mechanism of labor for a left occiput anterior position, in lateral view, which can be monitored in accordance with an exemplary embodiment of the invention;

FIGS. 7A-7F illustrate a normal mechanism of labor for a right occiput posterior position, in lateral view and in front view, which can be monitored in accordance with an exemplary embodiment of the invention;

FIG. 8A is a schematic diagram showing an exemplary birth monitoring system, mounted on a patient, in accordance with an exemplary embodiment of the invention;

FIG. 8B is a schematic diagram showing a detail of the attachments of internal sensors in the embodiment of FIG. 8A, in accordance with an exemplary embodiment of the invention;

FIG. 9A is a flowchart of an exemplary usage of the system of FIG. 8A, in a particular exemplary normal birth process, in accordance with an exemplary embodiment of the invention;

FIG. 9B is a time diagram of an exemplary abnormal birth progression, in which a monitoring system as described herein may be used in accordance with an exemplary embodiment of the invention;

FIG. 10 illustrates a second-stage detection device, in accordance with an exemplary embodiment of the invention;

FIGS. 11A-11G show traces and analysis for a first labor case, in accordance with an exemplary embodiment of the invention; and

FIGS. 11H-11L show observed three dimensional representation of cervix and head motion in accordance with an exemplary embodiment of the invention.

### DETAILED DESCRIPTION OF EXEMPLARY EMBODIMENTS

### State Based Monitoring Overview

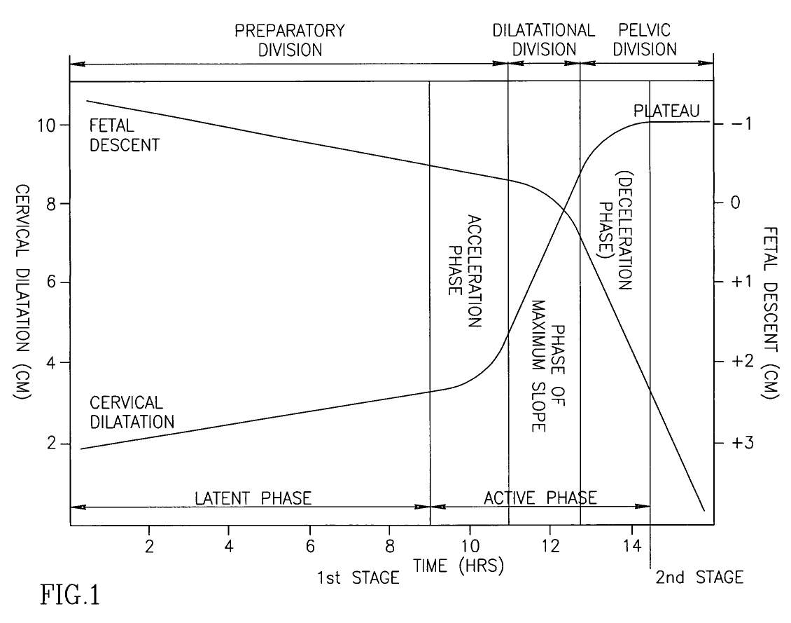

Referring back to FIG. 1, current practice is to chart the progress of a birth process along the graph of FIG. 1. As this graph is an average of many cases, a significant deviation must be detected if a caregiver is to be sure that there is a potential problem.

Even in non-pathological cases, this graph is problematic. As can be seen from the continuous and smooth nature of the graph, dilatation values cannot generally be used to identify the state of labor. Instead, an ongoing change or lack of change in dilatation values must be used (in the prior art). Further, as will be explained below, at large dilatation (>7 cm) the supposedly numerical values are actually symbolic values used to indicate an estimation of the labor progression. Thus, for example, even though an actual dilatation may be greater than 10 cm at maximum dilatation, an obstetrician will report “10 cm”. This results in a circular logic where an estimated state of labor is used to indicate a dilatation value, which is then used to show labor progression as compared to a standard graph such as shown in FIG. 1.

In an exemplary embodiment of the invention, the progress of a particular birth process is monitored by detecting the progression of the birth process from one state to the next state, non-progression to a next state and/or progression to an abnormal state. In an exemplary embodiment of the invention, the states are identified relatively quickly, for example in less than 20 minutes.

In an exemplary embodiment of the invention, anatomical changes are used to identify progression between different states of labor and/or identify a current state. In some cases, a statistical analysis of measurements is used to generate state information. In an exemplary embodiment of the invention, these changes are detected and/or measurements made using one or more position sensors mounted on a Cervical os. In an exemplary embodiment of the invention, relative changes between states are used and not just changes as compared to a standard graph. While the term “motion sensors” is used, it should be understood that various means may be used to detect relative and/or absolute position and/or orientation, for example, position sensors, transmitters, receivers, transponders and fiduciary marks detected using imaging. In addition, image processing may be used to identify key features on the Cervical os and/or fetal head.

In an exemplary embodiment of the invention, automated measurements, rather than, or in addition to manual measurements, allow data to be collected rapidly enough to give state information within a short time, for example, between 1 and 10 minutes. It should be noted that prior art methods are apparently hampered by one or both of slow measurement (e.g., manual measurement) and/or low discrimination caused by comparing data to an averaged graph. It should further be noted that due to natural variations, in some cases, the graph is wrong. For example, a labor can be effectively in the latent phase even with a large cervical dilatation (>3 cm). In another example, the second stage of labor can start with a dilatation smaller than 10 cm.

### Exemplary State Tree

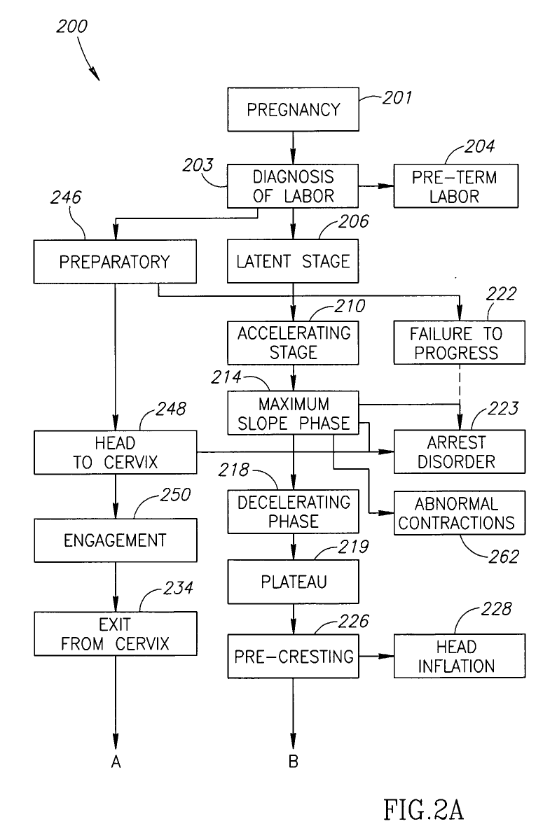

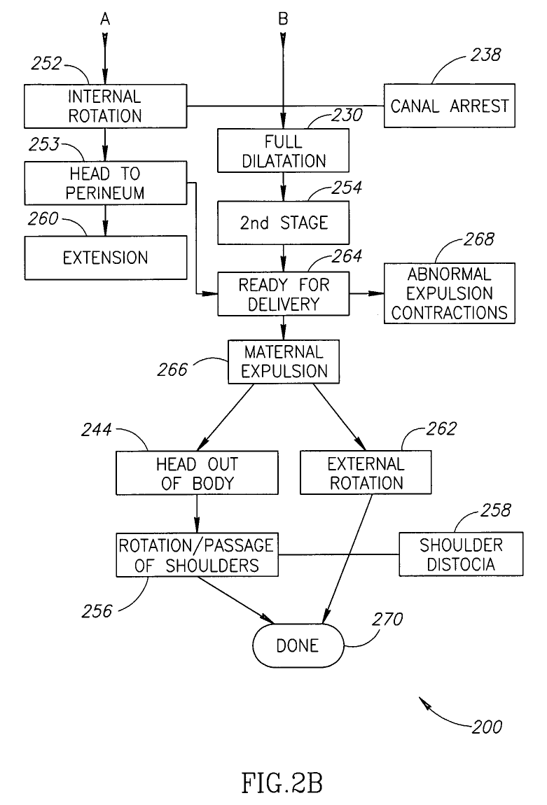

FIG. 2 is a tree-type state diagram 200 showing various states of a birth process (each shown as a box), at least some of which are identified in accordance with exemplary embodiments (methods) of the invention. Some states can be identified using multiple methods. It should be noted that the content (e.g., time duration, expected parameters) of each state may change depending on the situation in previous states.

In FIG. 2, two paths are shown, a path of the cervical state (center path) and a path of the head state (left path). Typically, the progression along the two paths is synchronized. However, they do not necessarily overlap exactly as shown in FIG. 1. This may be normal or abnormal. In some cases, a state is defined as a convergence of the two paths. For example, a ready for delivery state 264 defined as the 2^{nd} stage (254)/cervix being fully dilated (230) and head at the perineum (253). In one practical system, states are displayed as composite states including both head state and cervical state components. In other practical systems, the two paths are shown in parallel on a same display.

Following is a listing of such states with a short description of how such states and/or transit between such states can be identified. Some such identifications methods are elaborated in more detail below.

Pregnancy, 201, is the starting point from which labor or premature labor can be expected to start. One or more sensors may be implanted even before labor starts, for example, as a means to give warning for a start of labor.

Any labor-related activity may be analyzed as diagnosis of labor (203). Pre-labor may be identified if suitable sensors are attached before labor is expected. Typically, while contraction activity is identified, there is no/minor cervical dilatation.

In some cases, pre-labor leads to pre-term labor, 204. Optionally, pre-term labor is identified by a beginning of cervical dilatation before the fetus is of term. Optionally, the system tracks the effects of drugs or other treatment on arresting cervical dilatation and/or pre-term labor.

Once labor starts, there are two parallel states, one or both of which may be tracked, head state (on the left) and cervical state (in the center). In the following description, cervical state progression is described first. The two state types meet at a later time, in a normal labor.

A latent stage 206 is optionally identified by low variations in cervical dilatation (described below), low absolute dilatation and/or little or no head descent. In some cases, head descent occurs during the latent stage and so is not used for identifying this state. In an exemplary embodiment of the invention, latent stage identification is used to distinguish a “latent phase” stage from labor progression. Labor progression graphs that include the latent phase, as in the prior art, may appear flat and portray long labors and may erroneously yield early diagnosis of dystocia, with an associated mistaken intervention.

An accelerating phase 210 is optionally identified by intermediate variability in cervical dilatation. Optionally, a statistical test is used to detect the onset of this state, for example by analyzing a window of data to determine when cervical dilatation variability rate increases in a statistically significant manner.

A maximum slope phase 214 is optionally identified by a high variability in cervical dilatation and in head station. Typically, in the maximum slope phase values of CD variation is 0.75-3.0 cm and HS variation is 0.5-1.5 cm. Optionally, the maximum slope phase is recognized by analyzing the trace of the signals, which becomes more sinusoidal with minimal or no period of rest between consecutive contractions (e.g., a duty factor >0.3). Optionally, abnormal contractions at this stage are detected as an abnormal contraction state 262. In an exemplary embodiment of the invention, good/normal contractions during an active phase have a HS rise rate of between 1-4 mm/sec and typical decline rates of (0.5)-(2.5) mm/s. Typical CD rise rate is 1-6 mm/sec and typical decline rate of (0.5)-(4) mm/s. These are only typical numbers and other values may be common for other births. As noted, a template may be provided and such a template may include exemplary expected values, based on the general progression of the labor and/or patient information.

The onset of an optional deceleration phase 218 is optionally identified by a statistically significant change in cervical dilatation rate and/or by a reduction of cervical dilatation variation not accompanied by a decrease in HS variation. In some embodiments, a plateau state 219 is identified instead or in addition to deceleration phase 218, for example, based on a low change in cervical dilatation. Typically, the CD variation values drop by more than 50% to <1 cm while HS variation values are in the range 0.5-1.5 cm. It should be noted that when full dilatation approaches, the sensor noise may increase, for example, the sensors may move with the head. This depends, for example on location of placement, quality of attachment and where the fetal head is located. Optionally, such motion artifacts are detected and ignored, based, for example, on rotation of the sensors. It should be noted that one advantage of wireless sensors is that they lack an extension against which the head can push. Optionally, the sensors are positioned so that they do not move with the head. It is also noted that cervical sensors may be dislodged as full dilatation approaches. In some cases, the sensors will rotate with the fetal head. This may be prevented using suitable location and/or attachment of the sensors. Alternatively or additionally, such rotation is detected (e.g., during a contraction) and used as a measure herein.

A failure to progress state 222 (error states are shown on the right side of FIG. 2) is optionally identified by lack of change in head station even as cervical dilatation rate is variable. In particular, even if variability in head station is seen, this variability may be asymmetric in that only retrograde motion of the head is presented and not forward motion. Optionally, failure to progress is determined where there is no net advance over a period of time (e.g., at a same part of the contraction cycle, for example between contractions or at a peak of contraction). Retrograde motion and/or reduction in HS variability are optionally considered to indicate a problem. It should be noted that failure to progress can appear in different points throughout the labor. It can be identified by insufficient net progression of either head station or cervical dilatation in the presence of CD variations and/or HS variations. In an exemplary embodiment of the invention, the determination is based on the previous state of the labor, for example, if a maximum slope state has been detected a failure of progression would be a net progression of less than 0.5 cm in a period of 30 minutes. Asymmetry and symmetry in cervical dilatation and/or head station variations may be used for detecting other states or as an indicator for various conditions, for example head inflation.

Alternatively or additionally, an arrest disorder state 223 is optionally identified by considerable variability of HS and CD with no net progress in neither CD nor HS over time, for example 15-60 minutes.

A pre-cresting state 226 is optionally identified by the presence of small retrograde motion of the cervix and/or by reorientation of cervical sensors indicating they are pressed flat against the birth canal. In some cases, a partial full dilatation, when only a portion of the cervix tissue remains on the head, is optionally identified by asymmetrical cervical sensor level, e.g., retrograding of only one of the cervical electrodes. This may be reported as “9.5 cm” dilatation.

A head inflation state 228 is optionally detected by determining abnormal head descent past the cervix while full dilatation has not occurred. Alternatively or additionally, a suitable sensor (such as an ultrasound sensor that measures the distance to the skull) is attached to a fetal head to detect such inflation. It should be noted that this state (head inflation) may develop over time, for example being more pronounced during an engagement state than during a “head to cervix” state 248.

A full dilatation state 230 is optionally identified by a transit of the Cervical os over a crest of the fetal head. Optionally, such cresting is identified by detecting significant retrograde motion of the Cervical os towards the uterus, optionally not related to fetal head descent. In some representations, full dilatation state 23 and “2nd stage” state 254 are considered to be a single state.

A state of exit of the fetal head from the cervix, 234, is optionally identified from a relative motion of the fetal head sensor forward past the cervical sensors, for example, a motion greater than a length of the fetal head.

A state of canal arrest 238 is optionally identified when the head does not progress past a bend in the birth canal and/or if there is no or an incomplete rotation.

A state of internal rotation 252 is optionally identified based on a change in the orientation of the fetal head relative to the axis of the mother's body. Optionally, information about the initial position is used to determine the orientation in which dimension change should be noted. In this state, a progression of the head in a certain direction is expected and optionally monitored. Optionally, the beginning and end of state 252 are identified as separate states.

A state of head-exiting out of the body 244, is optionally identified visually. This state is typically followed by an external rotation state 262, also visually identified.

Referring back to diagnosis of labor state 203, a next head state is a preparatory division state 246, optionally identified by the head being high relative to IS, the head level being above cervix level <−2 and no correlation between head descent and CD. Optionally, head states are identified using positions relative to the cervix. In addition, in the preparatory phase values of less than 0.5 cm for CD variation and less than 0.25 cm for HS variation may be expected.

“Head to cervix” state 248 is optionally identified by head level is between −1 and +3 relative to cervix level. An optional useful parameter which varies during this state is a “strength of contraction”, which can be quantified using correlation of CD/HS variability (described below).

In some cases, fetal arrest (223) or failure to progress (222) happen before a “head to cervix” state. Head inflation state 228 may be identified after a “head to cervix” state.

An engagement state 250, is optionally defined by the BPD passing the pelvic inlet. It should be noted that in some normal birth situations, state 248 happens after the engagement.

Internal rotation state 252 may be identified, for example, by a change in orientation of the fetal head or by a change in a motion vector of a fetal head, even during a contraction

A state which can be identified manually is a head to perineum state 253, in which the fetal head presses against the Perineum, possibly being palpable from outside. This state may occur before, after and/or in parallel with an extension state 260. It should be noted that in general, the order of some states may vary between women. However, certain orders are not allowed (and indicate failure states). A flexion state (not shown) may also be defined. Lack of reaching this state generally indicates an abnormal condition.

After or before initial rotation 252, a 2nd stage state (254) is optionally identified by full dilatation (230) and in some representations, also engagement (250). Optionally, full dilatation is identified by the head having passed the cervix, for example, the head level being more than +3 relative to the cervix, for a cervix dilated at least 7 cm (otherwise it may indicate head inflation (228)).

In an exemplary embodiment of the invention, once both head to perineum 253 and 2nd stage 254 are identified, a state of ready for delivery 264 is identified. At this state, various medical preparations for birth may be made. A maternal expulsion state 266 follows where the mother is instructed to push out the fetus. This state optionally occurs once suitable expulsion contractions are detected. Abnormal expulsion contractions are optionally identified at an abnormal expulsion contractions state 268. Typically, normal expulsion contractions have a HS change magnitude of at least 1.5 cm, with a typical rise rate of 5-20 mm/sec, and a typical decline rate of (3)-(15) mm/sec.

A state 256 indicates rotation and/or passage of shoulders through the cervix. Optionally, this state is detected by the changes in cervical dilatation and/or motion of individual cervical sensors, after the head has passed. A potential complication is Shoulder Distocia 258, which is optionally detected by changes in cervical dilatation without an associated movement of the fetus, indicating that the shoulder is caught at the pelvic entry and is moving relative to the cervix during contractions.

Once the baby is born, a done state 270 can be manually identified. Optionally, the monitoring system is used to monitor after-birth processes, such as contractions and expulsion of the placenta.

### System Overview

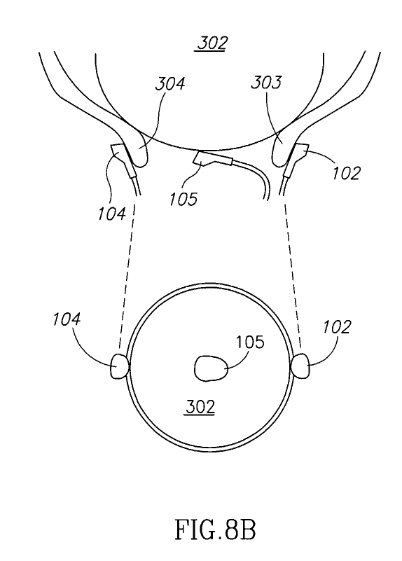

FIG. 8A is a schematic drawing of a birth monitoring system 100 and a mounting of sensors outside and/or inside the body, in accordance with an exemplary embodiment of the invention. FIG. 8B shows the mounting of sensors inside the body in accordance with an exemplary embodiment of the invention.

While a more detailed treatment is provided below, in an exemplary embodiment of the invention, system 100 includes one or more Cervical os sensors 102 and 104, an optional fetal head sensor 105 and a controller 101. Optionally, a display 116 is provided. A user input 128 is optionally provided for data entry. Optionally, one or more connections to external equipment are provided, for example a connection 115 to and from a fetal monitor. An optional remote unit 122, described below may be provided. A pair of reference position sensors (or transmitters or receivers, depending on the position detection system) 106 and 107 are described below.

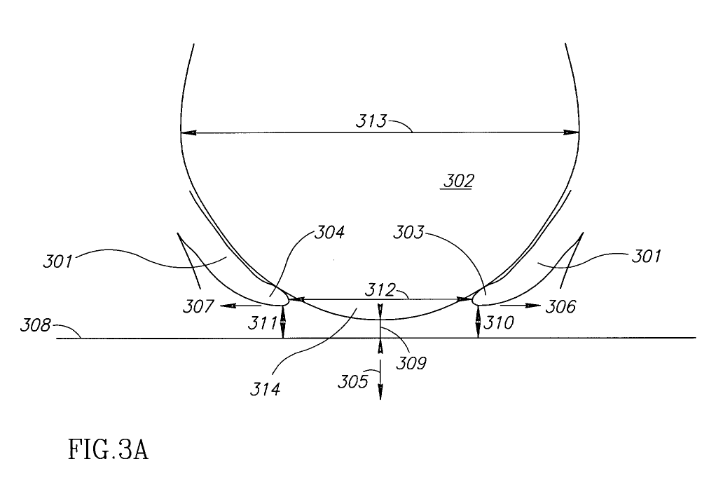

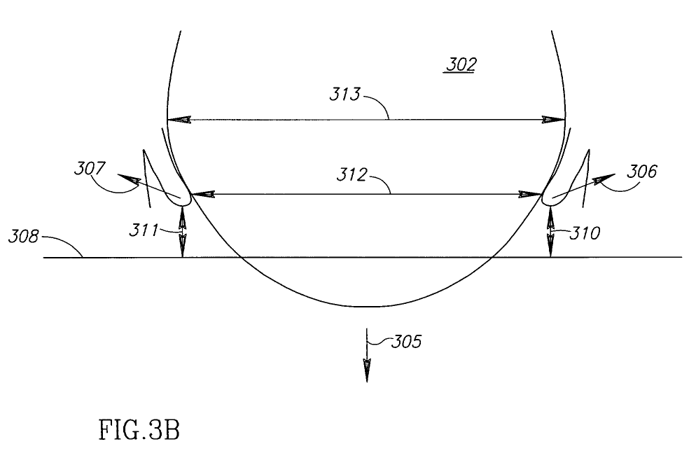

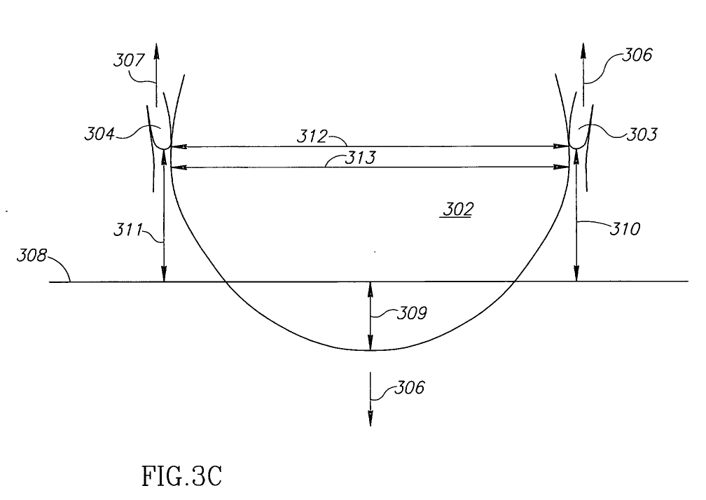

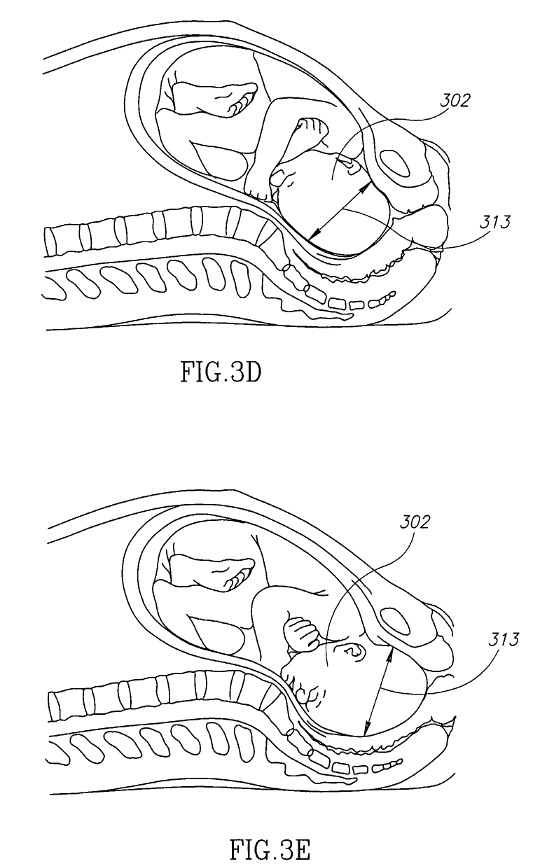

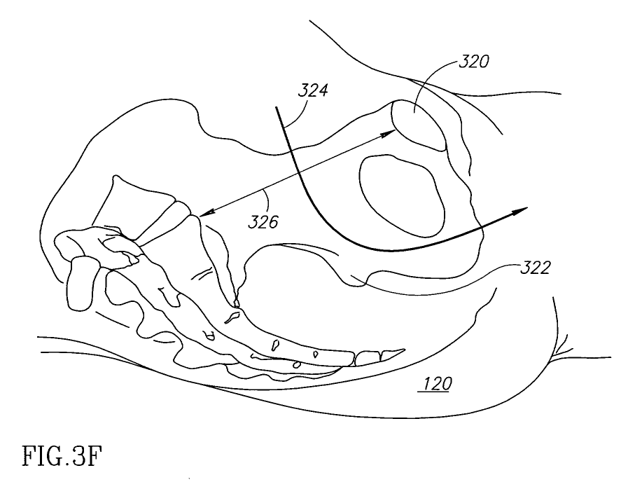

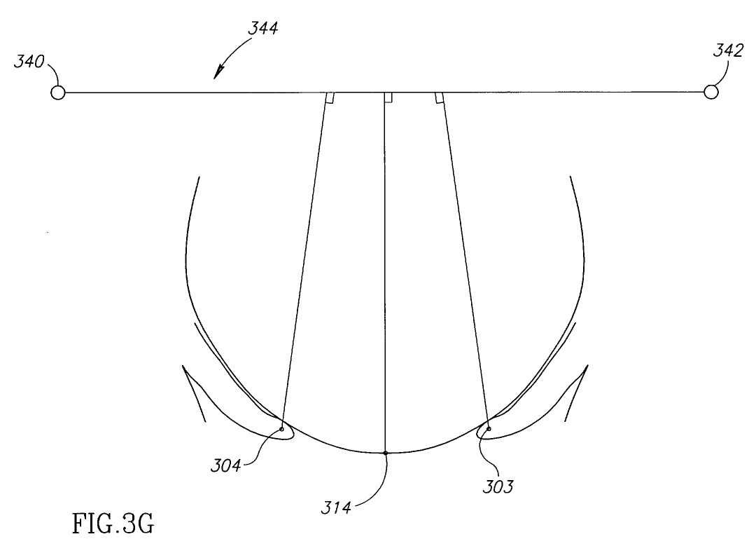

Cervical sensors 102 and 104 are shown attached to a lip 303 and a lip 304 of cervical external os 301 (see FIG. 3) at 3 and 9 o'clock, respectively. Other positions may be used as well, for example 6 and 12 o'clock or three sensors at 4 and 8 and 12 o'clock. In an exemplary embodiment of the invention, the sensors and/or attachment methods used are wireless or wired sensors, for example, of a type described in WO 02/098272, WO 02/98271, and U.S. Pat. No. 6,270,458, the disclosures of which are incorporated herein by reference.

A fetal head reference sensor 105 is optionally attached to a fetal head 302. These sensors and/or additional sensors may be used to collect physiological information about the mother and fetus, for example as known in the art. For example, one or more of the following can be measured: fetal HR/ECG, maternal HR/ECG, SpO₂, intra uterine pressure (IUP), blood pressure and/or TOCO.

Optionally, system 100 includes a storage unit, for example including a database, for example for archiving medical data, for storing patient data and/or for storing exemplary birth progression profiles. Alternatively or additionally, a hospital medical information system may be used for some or all of this information.

In an exemplary embodiment of the invention, system 100 provides one or more of the following functions:

(a) Monitoring. In an exemplary embodiment of the invention, system 100 is used to track the progress of a labor process and provide a local and/or remote indication of the current state and/of various physiological parameters.

(b) Diagnosis. In an exemplary embodiment of the invention, system 100 provides a tentative diagnosis of certain states, for example, pathological states.

(c) Warning. In an exemplary embodiment of the invention, system 100 is used to provide advance warning of possible complications and/or an alert that a certain state has been reached (e.g., second stage of labor), necessitating caregiver presence.

(d) Decision-making support. In an exemplary embodiment of the invention, system 100 provides information in a manner, which assists a caregiver in making a decision about what care to give, if at all. In a particular example, a state diagram is provided which tracks the states expected by the attendant and/or patient during a treatment for an abnormal condition, including, for example, warning indicators and suggestions for tests.

(e) Prognosis. In an exemplary embodiment of the invention, system 100 is used to predict a prognosis, including, optionally an expected time frame for one or more future states.

(f) Control. In an exemplary embodiment of the invention, system 100 is used to control the titration of drugs or other treatment to patients in labor, for example, stopping or preventing titration if certain states are detected.

(g) Management of multiple birth processes. Optionally, system 100 generates a warning if a same physician or attendant is expected to be required at two locations at a same time.

(h) Documentation and archiving. Optionally, system 100 includes an authentication module which allows and/or requires an attendant or possibly a particular attendant such as a physician identified using standard means, to authorize any information provided by the system for further use.

(i) Patient management. In an exemplary embodiment of the invention, system 100 is used to track instructions given to the patient. One type of instruction is to change a posture (e.g., to prevent a reduction in FHR). Another type of instruction is to push. Optionally, the system identifies effective expulsion contractions based on their shape (e.g., rise rates, decline rates, magnitude and/or duty factor) and generates instructions to the mother (e.g., using a speaker) to push and/or stop pushing. Optionally, system 100 keeps track of the physiological effect of the instructions.

In an exemplary embodiment of the invention, the use of system 100 allows a lower load on caregivers, whose task can be event driven (e.g., based on a system alert) or planned (e.g., based on a prognosis), rather than ad-hoc as a result of periodic manual examinations. Optionally, system 100 includes a workload planning module which estimates expected need for certain types of caregivers and/or equipment and provides a possible timetable for their use.

In an exemplary embodiment of the invention, system 100 includes a database of exemplary labor situations. Optionally, a current labor process is analyzed by matching it with a previous labor process. Alternatively or additionally, labor processes for a same patient can be compared. Optionally, even normal labor processes are categorized into 2 or more types, for example, 3, 4, 6 or more types, for example, based on one or more of age, weight, number of births, number of pregnancies, number of abortion, gestational age, size of fetus, ethnic origin, pelvic shape of mother, demographic data, previous CS, history of medications gestational age, expected confinement day, gravidity, parity, therapeutic abortions, spontaneous abortions, ectopic pregnancy, preterm, living children, mode of delivery, pre-pregnancy weight, current weight, height, temperature, heart rate, systolic blood pressure, diastolic blood pressure, US during pregnancy, estimated fetal weight, pregnancy abnormality, bleeding, infections, surgical interventions, medical history, family history, past surgical history and/or surgical category. Optionally, the time and/or dosage and/or increase or decrease of oxytocin and/or pitocin and/or other drugs and the reason for increase/decrease, if labor was induced, and maternal temp in 2nd stage or postpartum are also provided.

In an exemplary embodiment of the invention, system 100 includes an expert system that carries out the above functions based on predetermined rules. Optionally, new rules are defined based on data monitored by the system. Optionally, the system uses a heuristic approach to “learn” different users (e.g., obstetricians) and takes this information into account in the algorithm. For example, user systematic errors in sensor mounting can be identified and/or corrected for this way. In another example, system 100 compares and corrects for the bias between the user manual examination and the system results. As a result, system 100 can display numbers calibrated to what the doctor is used to seeing (e.g., 8 cm CD when there is really only 7 cm CD) and not feel a discord between his estimates and the system estimates. Different users may have different corrections applied (e.g., a typically over-estimating doctor can see 9 cm CD in the above 8 cm CD case).

### Detection of Crest Transiting (Cresting)

FIGS. 3A-3E describe a method of detecting the cresting of fetal head 302 by a Cervical os 301, in accordance with an exemplary embodiment of the invention. This cresting corresponds to states 226 and 230 (FIG. 2).

FIGS. 3A and 3B are schematic illustrations showing the relative position of Cervical os 301 and fetal head 302, during dilatation. Opposing lips of Cervical os 301 are indicated by references 303 and 304 (of course, the Cervical os lip is typically a complete ring). A useful anatomical reference is a line 308 drawn between the Ischial spines (see FIG. 3F, below), on either side of the pelvis. It should be appreciated that these are idealized 2D illustrations and in a real birth, the configuration may not be symmetric.

During the dilatation process, a front section 314 of fetal head 302 advances in a general direction 305 and pushes apart (i.e., dilates) lips 303 and 304, in directions 306 and 307, respectively, so that the distance between the lips is a distance 312. Distances 310 and 311 indicate the distances of lips 303 and 304 from a reference line 308. A distance 309 indicates the distance between fetal head front section 314 and line 308. A reference 313 indicates a BPD of fetal head 302.

FIG. 3A shows the anatomical configuration while dilatation is less than 8-9 cm, e.g., through the maximum slope phase. During this phase, distances 310 and 311 tend to stay more or less constant, as the direction of movement 306 and 307 of lips 303 and 304 is generally parallel to line 308. As head 302 is above line 308, this is referred to as a negative birth station.

In the prior art, a physician inserts his hand (or two fingers) into the vagina and spreads his fingers so he touches lips 303 and 304 simultaneously. The doctor estimates dilatation 312 based on the finger spread.

FIG. 3B shows the start of the pre-cresting state (which can generally correspond to a “deceleration” phase, albeit being measured in a different manner). Head 302 is past line 308, so this is a positive birth station.

In the prior art, a physician is supposed to again insert his (or her) two fingers into the vagina and spreads his fingers so he touches lips 303 and 304 simultaneously. However, this is not generally possible, as head 302 is in the way. Instead, the physician typically touches one side and then the other and tries to estimate distance 312. Another technique is apparently feeling the remaining length of lips 303 and 304 and subtracting that from the value of 10 cm. This method is incorrect, as will be shown below that it is known that lips 303 and 304 have a non-zero length even at full dilatation. Thus, in general, the physicians use the dilatation value in cm as an indication of how far along they feel the labor has progressed and not as an actual measurement that indicates a real progress. Further, as can be appreciated, when BPD 313 fits through the cervix, the actual dilatation will depend on the fetal head shape, which is a variable and not uniform for all fetuses. In particular, attendants use the code “10 cm” to indicate full dilatation. Very often, numbers between 7 and 10 cm are used to indicate how far away the goal of full dilatation is, and not actual dilatation. “9.5 cm” is sometimes used as a code for partial full dilatation, in which one side of the cervix is dilated and one side is not.

Also shown in FIG. 3B is that direction 306 and 307 of movement of lips 303 and 304 change and include a reverse component (retrograde), away from line 308. In an exemplary embodiment of the invention, this reverse movement, which increases distances 310 and 311, is used as an indication of change between the phases. Optionally a threshold value is used to determine that distances 310 and 311 have actually grown, for example, a threshold of retrograde movement of over 1 cm. Retrograde measurement below 1 cm is optionally assumed to be noise. Other thresholds, such as 0.5 cm can be used as well. Alternatively, the “descent” of the fetal head relative to the level of the cervix can be used.

FIG. 3C is schematic illustration showing cresting of Cervical os lips 303 and 304 over fetal head 302, at maximum dilatation. If a physician were to feel for lips 303 and 304, he would pronounce them “gone”. However, they are there, just past BPD 313, so they cannot be felt. It should be noted that in this example, full dilatation is accompanied with fetal head descent below the Ischial spines, however, this is not essential. Optionally, the indication used to decide is the retraction of the cervix towards the uterus.

At maximum dilatation, lips 303 and 304 transit past BPD 313 (typically including significant absolute motion of the lips relative to line 308). While this can happen in a short time, in some cases, it may take several minutes (or more). Further, cresting of different parts of the cervix may take place at different times. Once all parts of the cervix have crested, the full-dilatation state (230) is completed and head exit from cervix (234) starts. In an exemplary embodiment of the invention, a threshold value is used to detect the “full dilatation point” based on the large movement of lips 303 and 304 relative to head 302. Alternatively or additionally, there may be retrograde motion of the cervix os relative to reference line 308, which retrograde motion may also be used to determine the full dilatation point. Alternatively or additionally, what is detected is a change in direction 306 and 307 to include mainly a retrograde component and little movement parallel to line 308. Possibly, what causes the large retrograde motion of the cervix is that head 302 is no longer forcing the cervix forwards, so it can relax backwards. A smaller degree of retrograde motion can be seen in FIG. 3B, possibly due to the shortening of lips 303 and 304. Alternatively or additionally, what is detected in FIGS. 3B and/or 3C is that lips 303 and 304 lie flat against the birth canal. This may be detected, for example, using orientation sensors attached to the lips, in sensors 102 and 104. In some cases, only a single sensor is used to detect this flattening.

Optionally, the degree of increase in distances 310 and 311 during the stages of FIGS. 3A-3C, is used to assess a degree of excess cervical tissue. This may be used, for example as an input to future manual examinations (e.g., in future births) or to educate caregivers. Optionally, in this estimate, it is assumed that lips 303 and 304 will have some extent in the forward direction, even at maximum dilatation.

FIGS. 3D and 3E are side cross-sectional view of a birth process, showing cresting of Cervical os 301 over BPD 313. Again, it can be seen that lips 303 and 304 remain in existence also at maximum dilatation. The details of the slipping of the lips over the head are not shown, but as noted herein, the slipping may be asymmetrical, with some parts slipping first and others later.

Optionally, head angle and/or flexing are used to assist in determining the progress from state 230 (full dilatation) to state 234 (head exit), for example to compensate for misidentification caused by asymmetrical cresting. Optionally, such asymmetrical cresting is detected from the relative locations of position sensors on the cervix and used as an indicator in the progress of cresting.

It should be noted that in some embodiments of the invention, a geometrical connection is assumed between cervical dilatation and head station. Thus, full dilatation may be identified if head station is advanced enough relative to the location of the cervix. Some abnormal conditions, for example head inflation, may also be detected in this way, for example, head descent when the distance between the cervical sensors is clearly too small for head descent.

To complete the presentation of pelvic geometry, FIG. 3F shows a cross-sectional view of a pelvic region of a mother patient 120. Landmarks which are optionally used in calibrating the systems are an Ischial spine 322 and a symphysis pubic bone 320. With relation to the shape of the birth canal, an obstetric conjugate 326 and a pelvic axis 324 are marked.

In an exemplary embodiment of the invention, three cervical sensors are used so that a cervical plane can be defined. However, it may be undesirable to use more than one or two cervical sensors.

In an exemplary embodiment of the invention, a geometric reference is used in the direction of expected motion of the fetus and sufficiently far away. Movement relative to this reference is used to decide on relative positions of the fetal head and the cervix. Thus, no plane, per se, needs to be determined. The reference may be, for example, several CM away and in an angular range of within 30 degrees of the perpendicular to the cervical opening. In an exemplary embodiment of the invention, the reference is simply the line connecting the Ischial spines or a single Ischial spine sensor.

FIG. 3G is a schematic illustration showing the extraction of relative cervical and fetal head positions using a remote reference in the form of a line connecting the ASIS, in accordance with an exemplary embodiment of the invention.

In this example, a line 344 connecting two ASIS 340 and 342 is used as a reference. Alternatively, a different reference may be used, for example, a single sensor attached to the body at an intersection between a line coaxial with the initial path of the fetal head and a body surface. Optionally, the direction of this path is estimated from images of the maternal anatomy or from the direction of head descent during contractions.

A distance 346 is determined between a cervical lip 304 and line 344 (or other reference). Similarly, distances 348 and 350 are determined for lip 303 (optional) and fetal head front section (314). The relative descent of the fetal head and the cervix is optionally estimated by comparing distance 350 to the average of distances 346 and 348. In some applications, merely tracking distance 350 may be sufficient to determine a useful head station. In some applications, two or more reference sensors are used, one sensor for each segment of the expected fetal head path which has a different vector in space.

In an alternative embodiment, even if line 344 is known or knowable, distances to a single point on the line, for example its center, are used as a reference. Optionally, a position of an arbitrary point, for example a point inside the birth canal is used as a mathematical (calculated) reference point, to which the positions of the various sensors are compared.

In an alternative embodiment, a plane is defined connecting the fetal head (or one of the cervical sensors) and the ASIS. The projection of the other sensor positions on the plane is used to determine the appropriate relative spatial position.

It should be noted that in some embodiments, there is no need for 3DOF (3 degrees of freedom) sensors, rather, a one dimensional sensor may be suitable for providing some of the information. A second dimension of detection may be useful for determining the distance between the cervical sensors, for example, by directly determining a TOF (Time of Flight) between them.

In an exemplary embodiment of the invention, an estimation of effacement is obtained, at least during a state where there is a small cervical dilatation (<3 cm, for example), by determining the relative head descent between the fetal head sensor and the cervical sensor(s), optionally using a remote reference point or line, as described above.

### Head Inflation

Referring back to FIG. 3C, in some cases, distance 309 might be incorrectly assessed. In one example, the fetal amniotic sac may have not burst and the fetal head sensor is actually attached to the sac. In this case, an imager (e.g., abdominal or pelvic) may be used to determine if the fetal head position is as reported. Alternatively, the fetal head sensor may include an ultrasonic distance measurement sensor that determines the distance between the sensor and the head.

In another example, head 302 may experience inflation, in which fluid collects between the scalp and the skull. In an exemplary embodiment of the invention, inflation is suspected if the head station increases while cervical dilatation is not completed. Alternatively or additionally, a suitable sensor may be incorporated into fetal head sensor 105, to detect such inflation. One example of such a sensor is an ultrasonic transmitter/receiver which measures the distance from the sensor to the skull, by TOF measurement of reflection of waves (e.g., echoes) from the skull.

Optionally, a head molding state (not shown in FIG. 2) is determined if no inflation is discovered using a skull distance sensor and an abnormal head station is found for a small cervical dilatation.

### State Identification by Cervical Dilatation Variability

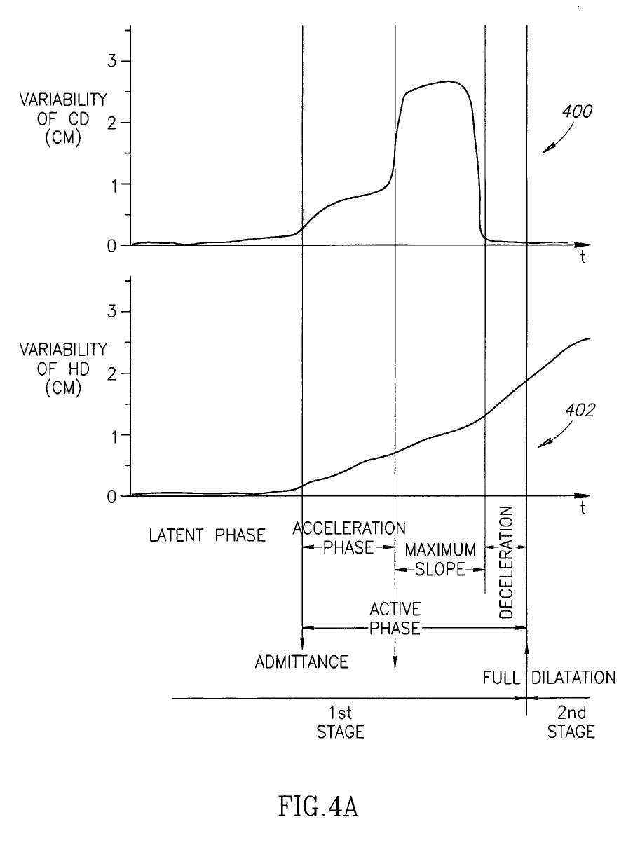

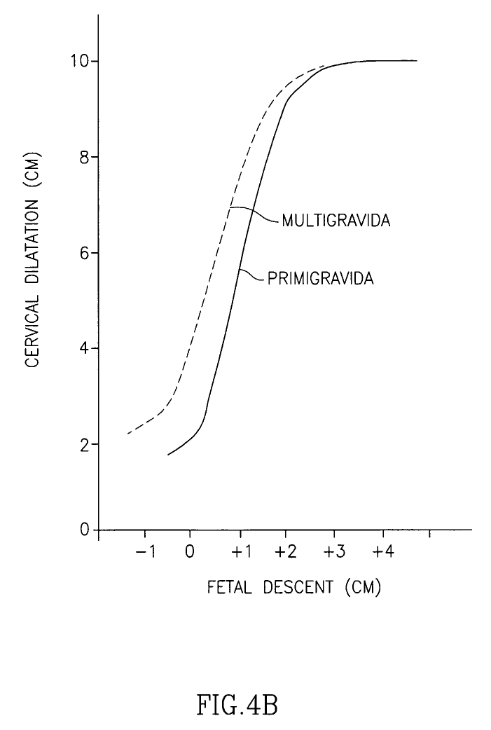

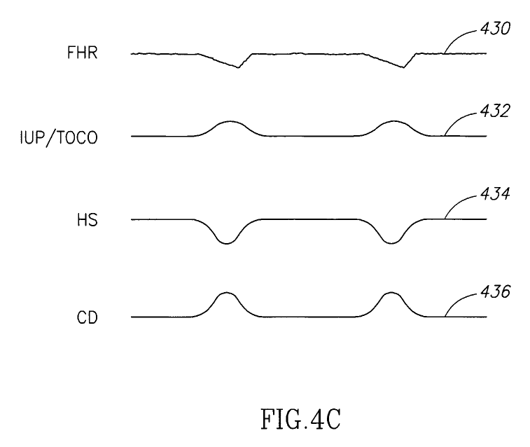

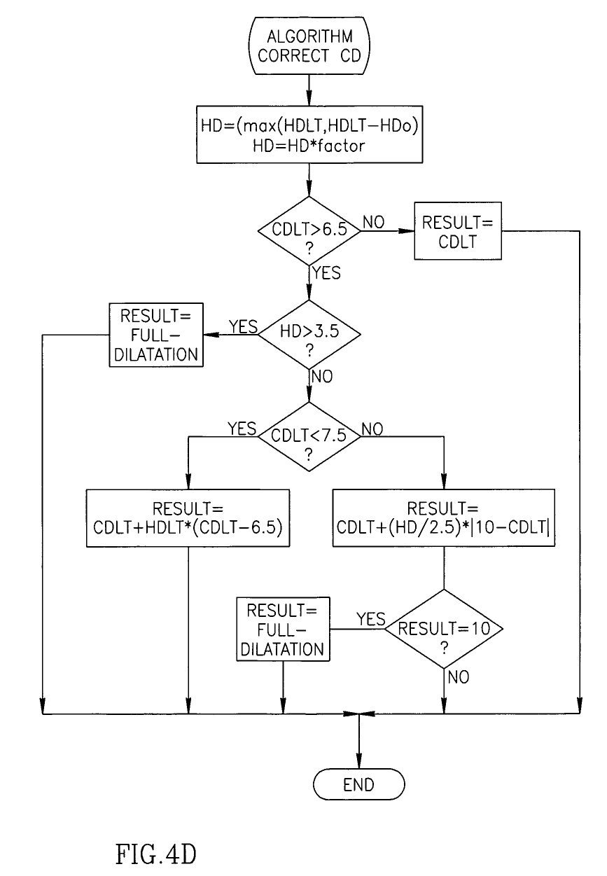

FIG. 4A shows two graphs 400 and 402. Graph 400 shows the variability of cervical dilatation as a function of the birth process progression, as determined from a plurality of measurements. Graph 402 shows a corresponding measurement for the variability of head descent. Both graphs are in cm. Graph 400 is based on measurements, while a part before active phase is estimated. It is possible that the cervix continues to vary in size after head exit In some embodiments, CD is re-referenced (or set) to zero after head exit, for better understanding of subsequent changes. Continued analysis, for example to detect shoulder passage may continue FIG. 4A is based on several tens of actually monitored births. Optionally, the two graphs in FIG. 4A are combined into a single graph of CD variability as a function of HS variability. As will be explained below, different methods of measuring and/or normalizing the variabilities may be used. Optionally, for example, variability is measured by peak amplitude detection (P) and then calculating P/T, T being time between peaks. Optionally, the ratio P/T averaged over several peaks, for example over 4 peaks, or alternatively averaged over a time interval, for example over 10 minutes. In general, variations may be counted on a time frame or on an event frame. In an exemplary embodiment of the invention, a parameter that is of interest to a physician is generated using the last 10 or 5 contractions, for example, or the recent 30 or 20 minutes. Such a parameter may be used as a predictor or to better understand the labor.

During contractions the degree of both cervical dilatation and head station may change considerably. These changes can be characterized by various parameters such as: change in amplitude (up to 2-3 cm in CD and up to 3-4 cm in HS), duration, duty factor (e.g., percentage of time amplitude is above 0.25 cm), repetition, rise and fall time and/or rate as well as their synchronicity with other recorded events such as intrauterine pressure, FHR, TOCO, etc. However, the inventor has identified a pattern in the variability of the amplitude that is apparently correlated with the various states of labor. Other thresholds can be selected the duty factor calculation, for example a 25% threshold which is set to the 25% of the maximal value. The type of threshold selected may affect the duty factor calculated, as it is possible for the contraction to cycle continuously with no rest period (but with a low amplitude period) between contractions.

As used herein, variability means change in a value over a short period of time, as compared to a longer term trend, which is used as a baseline. For example, during a contraction the CD changes and this change can be compared to the temporally averaged CD value (which hopefully is increasing regularly, even if not at a uniform rate). The values used herein are generally provided in cm, with the effect normalized or averaged for several contractions. Variability can therefore be defined, for example, by comparing change over a short period of time to an average value over that time. An exact exemplary formula is described below.

In the ‘Active phase’, the ‘acceleration phase’ is associated with increase in cervical activity and in head descent (peak amplitude of change ranging 5-10 mm, repeated every 3-5 minutes). This can be seen as an increase in variability. Alternatively or additionally as noted above, the asymmetry between rise and decline rates is apparently smaller than in maximum slope. In some patients it may be greater. As also noted above, in expulsion contractions, possibly more symmetry is found between rise and decline rates.

The variability in cervix dilatation increases and approaches its maximum during the ‘phase of maximum slope’ (e.g., variability of 1-3 cm), whereas simultaneous increase, but to a smaller extent (e.g., of 0.5-1.5 cm) is observed in head station variability. The ‘Deceleration phase’ is characterized by a sharp decrease in CD variability. On the other hand, HS variability continues to increase throughout the ‘Pelvic division’ and delivery. The exact variability values may vary and depend, for example, on demographics, fetal size, fetal shape, fetal presentation, number of birth, birth pattern, body shape and/or on properties of a particular patient.

During the ‘Latent phase’, no measurements were acquired, however it is hypothesized that uterine contractions usually lead to minor or no change in cervical dilatation (several millimeters) and have negligible effect on head station. Possibly, head descent is not defined during the “preparatory division”, if the head is relatively high (“floating”) and not at the birth canal. Possibly, however, when the head is high, it can respond strongly to contractions until such time that it reaches the cervix, at which point variability will sharply decrease. In some cases, the head is engaged by the birth canal even in the latent phase. Possibly, however, due to the generally weaker and less frequent contractions of the latent phase, HS variability is expected to remain small therein.

Thus, it is expected that in most cases, the onset of the “head to cervix” state (dilatational division) is characterized with very low/no CD and HS variability and then progresses to a state with higher variability in both as cervical contractions become more effective.

Failure to show an expected increase in HS variability may be indicative of a failure to progress (222), in which case an abnormal situation may be flagged. The exact abnormal situation may be identified, for example, based on the phase of labor and/or other measured parameters.

In an exemplary embodiment of the invention, the following fact situation:
{considerable CD variability (1-2 cm), considerable HS variability (0.5-1.5 cm), little/no net progress (<0.3 cm), frequent contractions (3 per 10 minutes) for one hour}
   is used to determine a state of lack of progress.

In some exemplary embodiments of the invention, the short-term temporal variations (e.g., variability) in CD and/or HS, and/or other geometrical parameters are used to tune and/or establish norms by which a series of dysfunctional labor states are defined. Optionally, a time resolution of better than 40, 20 or 10 seconds and a spatial resolution of 3 mm or better are used to characterize effects before, during and/or after individual contractions. These characteristics can be expressed in terms of various statistical parameters such as variance, mean, duration, repetition, rise and fall time and/or synchronization with TOCO/IUP and are optionally displayed numerically and/or graphically. In an exemplary embodiment of the invention, positions are sampled every 20 seconds for a period of 10 minutes. Other, slower data acquisition rates may be used as well, for example, every 40 seconds or every 1 minute. Other sampling periods, for example, 5 minutes or 15 minutes may be used as well.

It should be noted that in some embodiments of the invention variability information is used without absolute information on the initial position of the cervix relative to the fetal head and/or other cervix portion. As a result, some embodiments can use fewer sensors (e.g., measuring variability of only one cervical sensor) or to forego at least some of a calibration stage. In particular, slowly occurring drifts, or sudden changes caused by motion can be ignored, by measuring variations over a mid-term time (e.g., larger than 10 seconds and smaller than 30 minutes).

In an exemplary embodiment of the invention, one of the following processes is used to extract variability and changes in head station and/or cervical dilatation.

In one example, a measurement signal of one of the geometrical/physiological parameters e.g. (head-station or cervical dilatation) is acquired. Then, a median filtering with a window of 10 seconds is optionally performed on the signal, for smoothing. Then, the smoothed signal is differentiated. Optionally, artifacts are removed based on a physiological reasoning, e.g., based on rate changes. Optionally, the thresholds for the median filter depend on the state of the birth. Artifacts may also be removed before differentiation or after integration. Then the signal is re-integrated. Optionally, the baseline is not restored.

A short term curve is then optionally generated by median filtering with a window of 40 seconds. A baseline is optionally generated using a rank filter, for example a 25%-median filter that selects a 25% value in the window being filtered, for example a 3 minute window. A shorter or longer window may be used, however, this window size will generally include at least one contraction and at least one rest period, as contractions are typically shorter than 1-1.5 minutes and rest periods between contractions are also typically of similar size (at <50% duty factor). Thus, this window size can insure detecting a rest period with a 25% rank filter. Rather than select a 25% rank, other ranks, such as 20% or 30% may be used. As can be appreciated, the baseline generally shows the progression. Subtracting the base line from the short term curve will yield the contraction pattern. Optionally, additional filtering, for example based on physiological reasoning is carried out. In one example, contractions greater than 2.5 minutes in duration (optionally with a threshold on effect, such as 0.25 cm change in CD or HS) are deleted. It should be noted that while scalar values are described as being processed, higher dimension data can be similarly processed, such as a 3D spatial location of a fetal head sensor. It should be noted that even though a baseline is constructed, this baseline might not match the measured baseline.

In another method, averaging the raw data over a period of 20 seconds yields the short-term graph, improves SNR and/or removes artifacts due to sudden movements and technical errors. Note that each contraction lasts about 50-60 seconds, therefore a high pass filter such as 20 seconds moving average does not obscure effects of a contraction. The raw measurements are averaged (e.g., using a moving window average, median filter or other filter) to produce a long term trend, for example averaged on a time period of 10 minutes or more (e.g., allowing at least 2 contractions per interval in an active phase). This long term trend is subtracted from the short term data to provide the variability information, as follows. Subtraction of the long term trend from the short term average excludes the net progression from the CD and HS lines. One way to quantify variability is by calculating the root mean square (RMS) of the contraction line. Another way is described below. Calculating and/or analyzing statistical moments can be carried out using methods known in the art. Other exemplary processing methods which may be used to obtain and/or analyze variability information, for example, wavelets or spectral analysis, as well as other filters to characterize the variability line.

This extracted information can be used, for example to determine states, as described above. Alternatively or additionally, it may be displayed. For example, the variability can be displayed in separate graphs of CD variability as a function of time, HS variability as a function of time and/or combined in the same graph, for example, superimposed or provided one as a function of the other, optionally with various guides (e.g. for birth stage) marked. On this type of display a mismatch between existing variabilities and/or ratios as compared to expected values for birth states may be easy to identify manually. Optionally, the graphs are shown together with other displays such as TOCO and ECG. In an exemplary embodiment of the invention, a state based display is used and in each state an indication is made of the values. Particular examples are described below.

### Head to Cervix State Monitoring

FIG. 4B shows a known average relationship between cervical dilatation and head station. In an exemplary embodiment of the invention, a similar relationship is used to automatically identify the division of labor. Optionally, while the relationship is considered, the exact numerical values and/or slopes are not treated as constants, to allow for inter-patient variability.

In an exemplary embodiment of the invention, the relationship of cervical dilatation to head station is used to determine transition from preparatory division to dilatational division (redefined and modified for some embodiments of the invention as a “head to cervix” state), and to detect the transition to the pelvic division. The slope of the graph, which is mild during the preparatory division, rapidly increases during the transition to the “head to cervix” state. It stays almost linear in this state, typically having a slight difference between multigravida and primigravida. During the transition to the pelvic division (typically corresponding to the deceleration phase, FIG. 1), the slope sharply decreases to zero at full dilatation. In an exemplary embodiment of the invention, the shape of the graph (e.g., the bifurcation points in the graph and the relatively linear slope section) thereof are detected using statistical processing of multiple measurements, to determine a change in slope. However, these points are optionally not expected and/or not constrained to be related to the points on the “standard graph”, as the determination relates to a particular labor rather than an average labor process. In an exemplary embodiment of the invention, the determination is made using a relatively large number of spaced-together measurements, thereby resulting in a fast determination of state.

In an exemplary embodiment of the invention, the graph of FIG. 4B, as extracted for a patient is used for tracking a “head to cervix” state. Numerical analysis of FIG. 4B may also be performed. For example, a slope of the graph indicates the compliance of the cervix to the head. Abnormal compliance values may indicate an abnormal state.

It should be noted, that being defined in functional and/or measurable terms, the “head to cervix” state can be more useful than a general “dilatational division”, defined in the prior art.

As noted above, a graph similar to 4B, but based on variability can be used in the same way as described above.

### Data Integration

In an exemplary embodiment of the invention, data from multiple sources is integrated. Optionally, the sources comprise at least one geometrical data source (such as provided by position sensors, e.g., CD and HS) and at least one physiological data source, such as fetal heart rate, IUP and/or TOCO. Also maternal physiological information may be used, for example as described below.

FIG. 4C is a schematic showing of real-time traces of four data sources, a fetal heart rate trace 430, an IUP and/or TOCO trace 432, a head station trace 434 and a cervical dilatation trace 436. Typically, IUP replaced TOCO at some stage of the birth, for example, once medication is provided. Real (measured) traces are shown below.

In an exemplary embodiment of the invention, the display of the multiple data sources is integrated such that multiple traces are shown and an operator analyzes them manually. Alternatively or additionally, an automatic analysis is performed, for example to determine bad states and/or dangerous conditions, for example as described below. Optionally, the traces of both geometrical data and physiological data are printed on a same paper, for example, by transmitting the traces to a standard physiological monitor with a built-in printer, or by providing a modified monitoring software. Optionally, the printing is as summary data, for example every few inches of paper. For example, numerical values and/or small graphs may be printed. Such small graphs may show, for example, past, present, expected and/or desired values. Alternatively or additionally, warnings and/or state indications may be printed. Optionally, one or more of the following information is printed in addition to physiological data (e.g., IUP, TOCO, FHR and/or maternal information): CD, HS and/or variability of one or both.

Another optional display type is a histogram-like display, which bins average peak amplitude change as a function of CD value, for different CD value ranges. Possibly, displaying peak amplitude is less effective than showing variability information in that the peak values or average peak values ignore the effect of the duty factor.

Other information may be shown instead of average peak amplitude. Alternatively or additionally, a different scale (set of bins), such as head station or time or state is used instead of cervical dilatation value. Optionally, the histogram shows an expected effect of a drug. In either case, the histogram can be used to compare expected values to real values. In an exemplary embodiment of the invention, the histogram or other concise data representation method shows data collected (and optionally processed) from a relatively long period of time, such as 2 hours or more, or less, such as 30 minutes or more. Raw data of such a time length is very difficult to assimilate. The partogram, while concise, is too concise in that it does not allow comparison to the patients own behavior (due to resolution) or to view important information, such as variations. It is expected in some embodiments of the invention, that a concise statistical display assists in detecting states.

Integration of data on a display may include, for example, correlation, determination of trends, comparison of phases and/or wave forms and/or other functional relationships.

Optionally, geometrical and physiological information correlation is used to determine if sensors are properly attached. For example, a very low TOCO while CD is changing considerably probably means the TOCO sensor is not attached properly. Similarly, a high IUP without any change is HS, may mean the fetal position sensors are not connected or being read properly.

In an exemplary embodiment of the invention, TOCO information is used as a trigger or as a window to define when to measure the efficacy and other contraction parameters.

In an exemplary embodiment of the invention, fetal Bradycardia is correlated with geometry to assist in determining its significance and/or possible cause. For example, a fetal heart variation that persists after CD is reduced may indicate an abnormality. Similarly, a correlation between fetal oxygen saturation and CD or HS may indicate a problem.

In an exemplary embodiment of the invention, IUP is correlated with geometry to determine a danger state of a fetus. For example, a norm may be set for HS change in relation to IUP. If, for example, a high IUP is not correlated with any change in HS (or only a small change) this may indicate a high pressure on the fetal head. Alternatively or additionally, such a correlation may show the effect of a drug therapy intended to slow or hasten labor by strengthening or weakening contractions. For example, if IUP goes up but geometry does not change, this indicates that the drug therapy may actually be dangerous.

In an exemplary embodiment of the invention, maternal problems are diagnosed. For example, changes in blood pressure or heart rate which correlate with changes in IUP and/or CD, can be indicative of maternal problems. A particular example is if CD variation goes down together with blood pressure over a series of contractions, this may indicate a weakening of the mother. This may be correlated, for example, with various parameters of the contraction, such as its length.

In an exemplary embodiment of the invention, IUP values are used to gate CD and HS measurement. For example, IUP is assumed to be less sensitive to motion artifacts. When the patient walks and a contraction starts as per the IUP, HS and CD are measured. Optionally, a patient is asked to stop moving at such a time, for example, by giving instructions ahead of time or by an audio signal from system 100 in real time.

In an exemplary embodiment of the invention, a triggered display is shown, in which the display starts with a contraction, for example as detected by IUP or EMG. Optionally, multiple contractions are overlaid. Optionally, the effects of the contractions are overlaid. Optionally, the effects are normalized, for example for length and/or amplitude. Optionally, variation ranges and/or other statistical information are shown, for example, as different densities.

### Contraction Shape

The inventor has discovered that the shape of a contraction (e.g., geometrical results and/or pressure values) can be analyzed and used for various purposes. In particular one or more of the following parameters may be of interest: rise rate, decline rate, duty factor and relative duration and/or synchronicity and/or phase delay of geometrical measurements compared to pressure measurements (IUP or TOCO) or fetal vital sign measurements (FHR and/or SpO2), in addition to or instead of frequency, area under the contraction curve and amplitude. In an exemplary embodiment of the invention, expected norm values for one or more of these parameters are used to detect abnormal states and/or to filter out artifacts. In general, thresholds used for filtering are not set to norm values but to greater values, for example, to physiologically possible values. As an example, some motion artifacts generate rise rates that are physiologically impossible. In another example, expulsion contractions with duty factor of under 0.1 are clearly abnormal.

In an exemplary embodiment of the invention, a contraction activity graph is generated by using a threshold of 0.25 cm to identify a potential contraction. The contraction activity is estimated from the contraction curves using a running average over 10 minutes and summing values exceeding the threshold. The obtained values were divided by 0.5 (representing a maximum duty factor of normal contractions). Possibly a different normalization value is used, for example, if a higher duty factor is found for some patients. It should be noted that this activity parameter is generally sensitive to both the magnitude and the rate of contraction (assuming maximum contraction length is pretty uniform between people). Optionally, cervical dilatation variability values and/or head station variability values are used for qualifying states and for assessing uterine work/effectiveness. Typically, in the accelerating phase values of 0.25-1.0 cm are seen for CD variability and 0-0.75 cm for HS variability. Optionally, the accelerating phase is recognized by its contraction curve with a duty factor (the ratio between contraction duration and interval between contractions) typically between 0.1-0.3.

### Contraction Usefulness Parameters

In an exemplary embodiment of the invention, the “usefulness” of a contraction is assessed. For example, one or more of the following measures may be used:

(a) Magnitude. This indicates the amount of work (or energy) in a contraction. If only contractions of a low magnitude are found, it may indicate a patient that is still in a latent phase. Magnitude may be estimated, for example, by IUP (e.g., by integrating pressure over time and optionally normalizing the value). EMG signals may also be used to estimate the amount of work carried out by the muscle.

(b) Efficacy. This indicates how much the particular contraction advanced the labor. For example, the change in cervical dilatation after the contraction. In an exemplary embodiment of the invention, the efficacy is tied to the stage of labor. For example, after full dilatation is reached, no further cervical dilatation is expected. Optionally, a correction for a progression of labor is provided. For example, indicating a normative unit change in cervical dilatation for each cervical dilatation value. As cervical dilatation reaches full dilatation, smaller net increase is expected each contraction, even if all contractions have a same efficacy. A similar correction may be provided for head station. Optionally, head station efficacy is measured separately from cervical dilatation efficacy. Alternatively, both are combined into a single efficacy measure. Optionally, different efficacy values are expected for different parts of labor. Optionally, an efficacy profile is expected for the birth as a whole. Optionally, labors are categorized based on an expected predictability thereof. In one example, if changes in variability match expected progression graphs (e.g., FIG. 4A), the labor is noted as being more predictable, at least in some phases thereof. Optionally, the relationship between CD variability and HS variability is used (e.g., using a look-up table or a neural network system or a rule based system) to generate an expected progress rate in c/hour. Optionally, the progress is also dependent of past performance. The relationship may be normalized.

In an exemplary embodiment of the invention, one or more “CLM numbers (computerized labor management)” which are indicative of the state and of the prognosis of labor progress under various possible scenarios are provided. These numbers can represent a function of measured parameters such as amplitude, rate and duration of dilatation descent and position, or can be evaluated by using pattern recognition methods or other methods with or without parameterization. Optionally, the parameters taken into account include all of CD variability, HS variability and duty factor.

(c) Efficiency. This indicates how efficient a contract is, and may be expressed as a ratio between the Magnitude and the efficacy. Efficiency going down (e.g., compared to an expected profile) may be an indication of an abnormal situation.

(d) Duty factor. This indicates how often and long the contractions are. As the maximum duty factor is a physiological limitation, lower duty factors can be used to indicate a birth state and/or an abnormal or inefficient situation.

(e) Rise/decline rates. Generally, the rise rate indicates a ratio between the strength of the contraction and the compliance of the tissue and/or fetus. A lower rate may indicate a less effective/useful contraction. The decline rate (e.g., of CD) may be used to indicate the resilience of the tissue and/or fetus and indicate, for example, if the cervix is effaced (less resilient), if there is head inflation (less resilient, greater range of motion). The decline rate (e.g., of pressure) may also provide an indication of the synchrony of activation of the uterus. If the decline is slow, this may indicate that parts of the uterus are contracting out of turn. Also a slow rise rate may indicate a problem with uteral synchronization.

(f) Delay (e.g., average, in rise, in peak plateu, in decline and/or quiet period) between various measures, such as pressure measurements and geometrical measurements or between different types of geometrical measurements. Certain delays above a threshold may indicate a disassociation indicating abnormal contractions or effects. For example, too large a delay between pressure and HS change may indicate that the fetal head is arrested. Conversely, if HS change stops long before pressure peaked, the additional pressure may be wasted.

(g) uEMG. In an exemplary embodiment of the invention, electrical synchronization of the uterus is used to assess if the contraction wave in the uterus is progressing properly. In an exemplary embodiment of the invention, the correspondence between electrical activity and result shows whether modifying electrical activity is needed and/or whether modification is having a desired effect.

Optionally, these measurements are determined as values and/or as variations of values.

In an exemplary embodiment of the invention, a contraction efficacy (value and/or variability) is determined. Optionally, contraction efficacy is measured when one or both of geometrical sensors and other sensors (e.g., TOCO or IUP) indicate a contraction is in progress.

In an exemplary embodiment of the invention, what is monitored is CD or HS variability as a function of IUP. In one example, when drugs are titrated, uEMG or IUP/TOCO signals can provide feedback on the effect on the muscle, while HS variability and CD variability show the effect on labor progression.

In an exemplary embodiment of the invention, what is identified is a maximum throughput state, in which it is determined that the uterus is doing its best. Overstraining the uterus at this point might cause damage and/or not make labor progress faster. In an exemplary embodiment of the invention, once a threshold of CD variation of over 0.5 cm is found and a duty factor of close to 50% is detected, this indicates that the uterus is operating at a peak condition, further titration of drugs may increase contraction strength but without progressing labor. In any case, once the uterus is doing well (even if labor is progressing slowly), it is generally advisable to allow the birth to continue naturally. Optionally, the progress rate can be estimated more precisely once this performance is found (e.g., by dividing remaining progression by progression rate. It should be noted that unlike using partogram, this identification does not depend on the CD. In an exemplary embodiment of the invention, drug titration is controlled to achieve this state of maximum effectiveness of the uterus.

This state can also be called an individual maximum slope, as it indicates that the patient is in a maximum slope (of a partogram of CD) while the slope is the maximal that can be expected for that patient. In an exemplary embodiment of the invention, a device is provided which includes a meter or flashing Led or a dedicated display, which indicates that a maximum slope state is approaching (e.g., and drugs should be stopped or reduced). Such approaching can be detected, for example, by the duty factor increasing regularly and the CD change being continuously above a threshold.

Optionally, a contraction efficiency is defined as a function of the change in CD and/or HS amplitude caused by the contraction and the HS (and/or CD) net progress (δ). In one example, the following formula is used δ/{sqrt(1+Dcd*1+Dhs)}, where Dcd and Dhs stand for change in CD and HS over a contraction.

Optionally, a contraction efficacy is determined for a plurality of contractions and/or for a time period (e.g., using a moving window method), and may serve, for example as an indicator of normal or abnormal labor processes. Variations in contraction efficacy may be normal or indicate a problem. Optionally, a baseline value is measured for a plurality of women as a function of labor stage and the actual value is compared to the baseline.

Optionally, determination of efficacy takes into account one or more parameters of the contraction, for example, width, pressure (internal or external), rise and/or decline rate, peak duration, peak amplitude and/or area under curve.

In an exemplary embodiment of the invention, one or more desired patterns of changes of the above measures are provided. In one example, the patterns are collected from the patient, for example, from a pervious birth for long term patterns (e.g., 1 hour and up) and from a current birth for short term patterns (e.g., 10 or 20 minutes). Optionally, different states have different values and/or formulas associated with them, for example, for stages where no cervical dilatation variation is expected and/or for stages where considerable head station change is expected. Optionally, a match with a known “good” or “bad” pattern is shown to an operator.

### Calibration and Initialization

In an exemplary embodiment of the invention, determination of state is used to assist in initial calibration of system 100 when attached to a patient at an unknown stage of labor. In one example, a graph of CD and HS variations collected over a period of time is matched to an expected and/or average graph, to assess a state of labor. Alternatively or additionally, the state is identified from geometrical and/or physiological considerations and monitoring is continued from that point. Optionally, a history is estimated after a plurality of current measurements are collected. For example, changes in head station can be collected and then localized in space once a calibration is performed.

Optionally, the cervical sensors are calibrated, for example using a phantom or a sensor mounted on a handle with a known geometry to emulate a fetal head. Optionally, the calibration corrects for sensor size and/or positioning. Optionally, a visual check is made to assess the need for correction (e.g., relative placement on the cervix). Optionally, the orientation of placement on the cervix is estimated from the rotation of the sensors during and/or between contractions. In an exemplary embodiment of the invention, a calibration as suggested in PCT/IL2004/001092 filed Nov. 29, 2004, the disclosure of which is incorporated herein by reference, is carried out. Optionally, the calibration comprises different calibration values for different body postures. Alternatively or additionally, the calibration comprises assuming there are no sudden changes in CD and/or HS and that any such sudden changes are due to movement or the like. Therefore, the values measured are constrained to be continuous (e.g., a continuous progression of baseline CD).

### Cervical Dilatation Correction

FIG. 4D shows a flowchart of a method of correcting an actual measured cervical dilatation to match a presentation method now used by physicians. In this flowchart, HD is calculated as NH3−(NC1+NC2)/2, where NH3, NC1 and NC2 are distances from the virtual line shown in FIG. 3G. HD0 is the HD during attachment of the electrodes/sensors. HDLT is the long term moving average of HD over 10 minutes. CDLT is the long term moving average of the distance between two cervical sensors, over a period of 10 minutes. Factor is empirically set to 0.7. Optionally, HD is a measured head station, relative to the level of cervical probes.

As can be seen, correction is optionally applied only after a certain point (CDLT>6.5, for example) and is optionally applied differently depending on the head station. Any result of dilatation over 10 cm may be considered full dilatation, in some implementations. Alternatively, a decrease in the distance (NC1+NC2)/2 compared to a baseline (NC1,0+NC2,0)/2 calculated at the beginning of the procedure, is the parameter used for CD correction. Optionally, using a single probe on the cervix, only the distance NC1 is used. In a similar manner, the correction will be applied after a certain point (CDLT>6.5, for example) with a different factor. It should be appreciated that these values may be varied without altering the scope of the method.

Optionally, the values of cervical dilatation and/or head station may take into account their variability due to contractions to correct their values, for example, by adding a certain factor to dilatation in the presence of significant changes during contractions. It is expected that this correction may lead to values closer to those reported by humans using digital manipulation, e.g., if there is a large variability, humans will tend to over estimate because the cervix is more flexible.

Possibly, there is a relationship between the cervical dilatation change during contraction and tissue consistency and/or compliance. Based on the assumption that there is a connection between the effect of applied pressure imposed by uterine contraction and by human fingers, e.g., not just variability but also strength of contraction, a better correction for the measured value may be provided. Optionally, the actually measured value is corrected so that when displayed it would match what a physician (or a particular physician) would have reported. Optionally, system 100 learns the individual offsets and idiosyncrasies (e.g., how hard doctor spreads fingers at each CD).

In an exemplary embodiment of the invention, consistency is estimated by applying a known pressure to the cervix os while measuring its distension. Optionally, such measuring is carried out during manual measurement. Optionally, a pressure sensor is worn on the physician finger to provide the pressure. Alternatively, a calibration per physician may be provided. Optionally, a device that stretches the cervix is used and when combined with measured distension used to estimate consistency.

In an exemplary embodiment of the invention, a correction for patient geometry is provided, for example, an obese person or a small person may have different baseline geometrical sizes (e.g., expected maximum cervical dilatation or threshold at which correction is applied). For example, the correction may be based on demographic information, previous births, manual measurement, consistency and/or other known or estimated parameters of the cervix.

Optionally, CD includes compensating for incorrect placement of probes or asymmetric expansion of the cervix. In one example, the vector motion of the probes is used to estimate their relative positions. It should be noted that detection of full dilatation will be correct even if the CD measurement itself is not.

### Geometrical Chances

In an exemplary embodiment of the invention, geometrical anatomical changes are used to detect a change in state.

In one example, mentioned above, referring to FIG. 3C, lips 303 and 304 of the Cervical os lie flat against the birth canal. This orientation of the lips can be detected using a sensor 102 or 104 that includes an orientation sensing ability.

In another example, once fetal head 302 passes the cervix, the cervix can contract. This contraction may be detectable in some cases and indicate passage of the head. Alternatively or additionally, the diameter of the Cervical os may change as the body of the fetus flows through, for example, the diameter might increase at the shoulders and reduce once the pelvis of the fetus passes.

Optionally, cervical effacement is detected by measuring the thickness of the cervix, for example as described above.

### Head Station in Second Stage

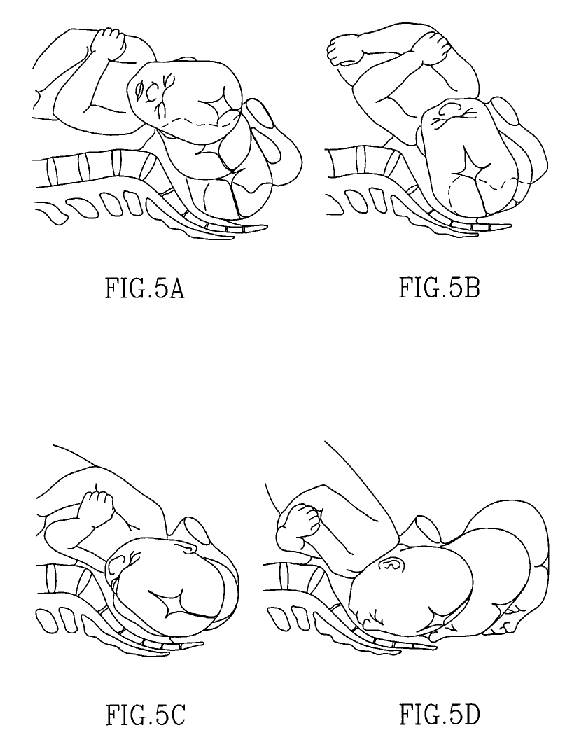

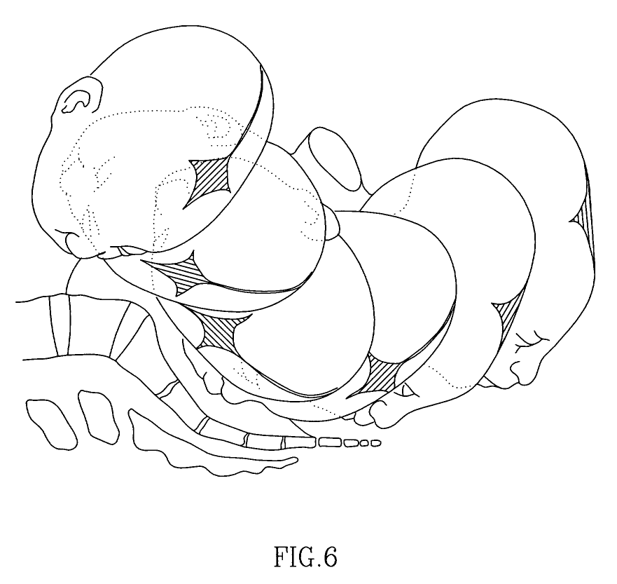

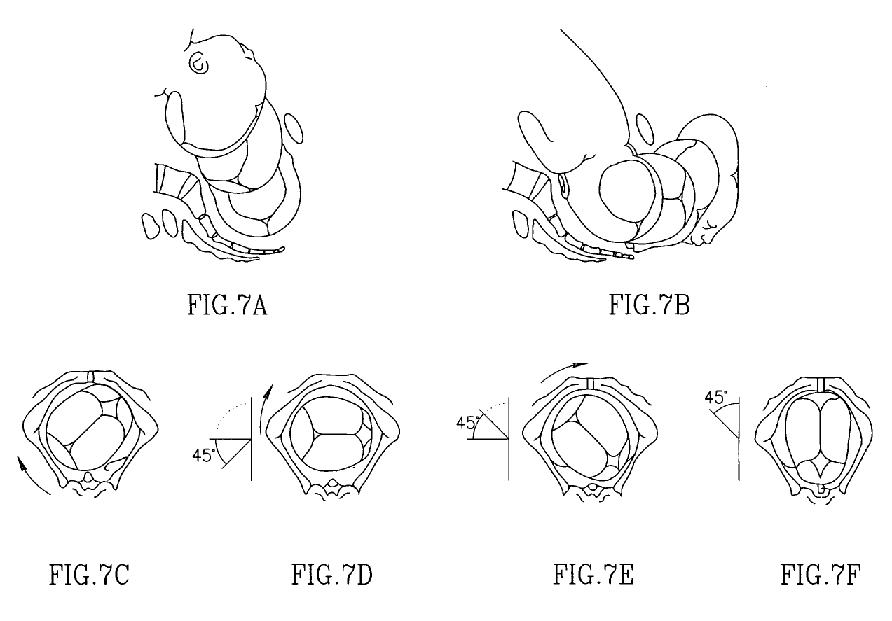

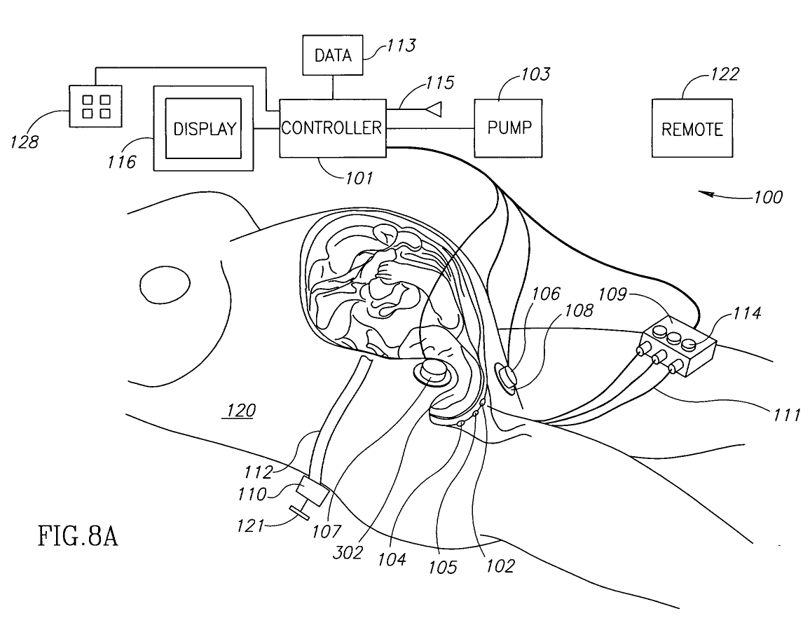

FIGS. 5-7 illustrate different birth mechanisms for different fetal presentations. However, what is common to all three is that during passage through the birth canal, fetal head 302 rotates and extends. For each presentation these orientation changes occur differently. However, these orientation changes are forced by the birth canal geometry.

In an exemplary embodiment of the invention, system 100 tracks the orientation changes, optionally matching the changes and their order to expected patterns of changes. In one example, a caregiver indicates (e.g., prior to labor or at full dilatation) what the fetal presentation is. Thereafter, system 100 uses a set of expected states based on that presentation. Optionally, the expected states include parameters for the states, for example, expected values for measurements, expected degree of rotation of head and/or expected duration of states (or possibly maximal reasonable duration).

In an exemplary embodiment of the invention, fetal head position is provided not in numerical terms, which may vary from patient to patient, but in functional terms, for example “before rotation engagement”, “before bend” and “after bend”. Optionally, the fetal head position and/or orientation are shown on an image or a graphical representation of the patient's pelvis. Optionally, an expected birth process is indicated as well. In one example, a generic representation is geometrically modified to match maternal measurements.

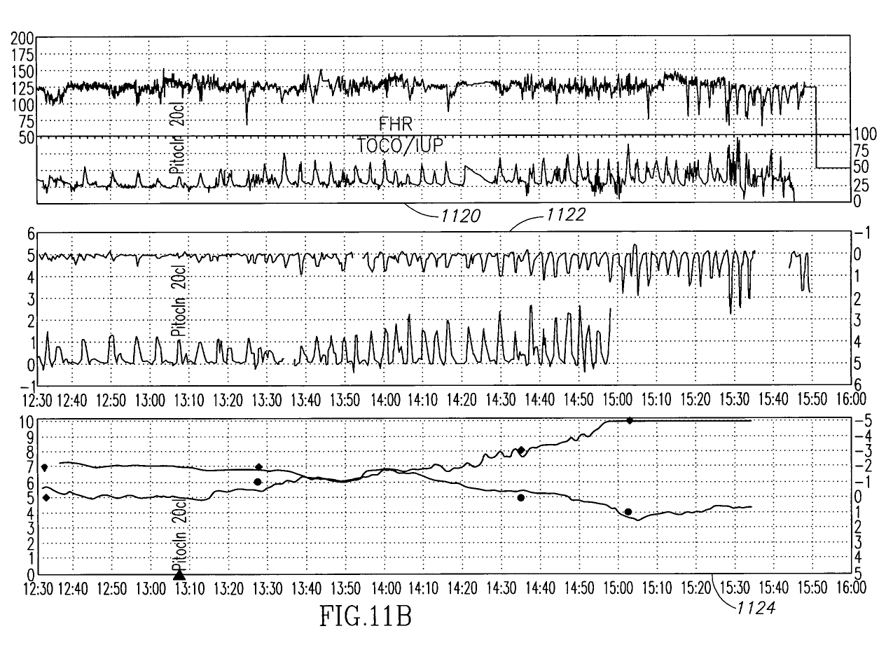

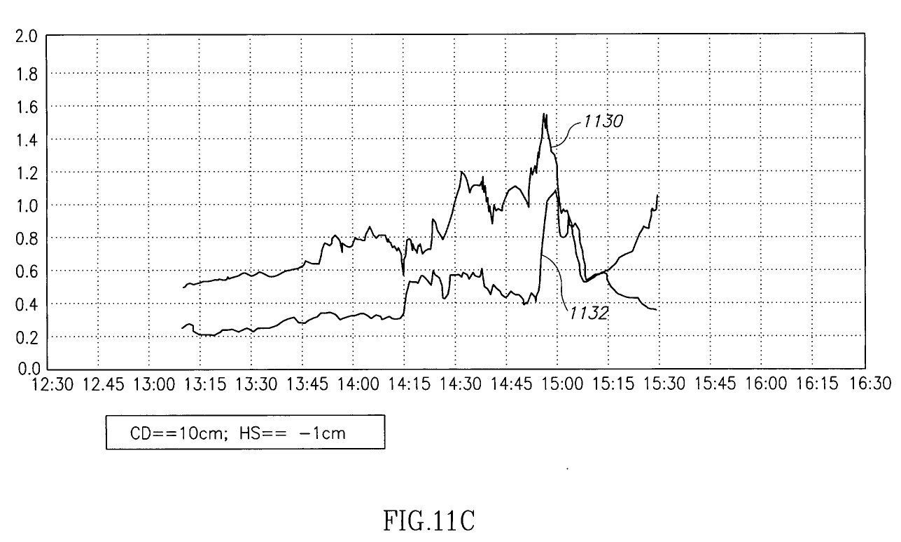

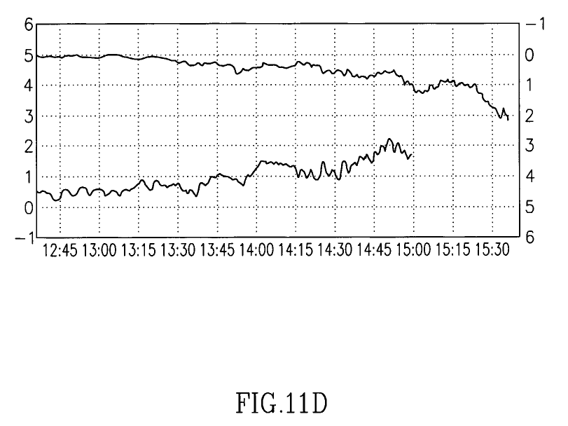

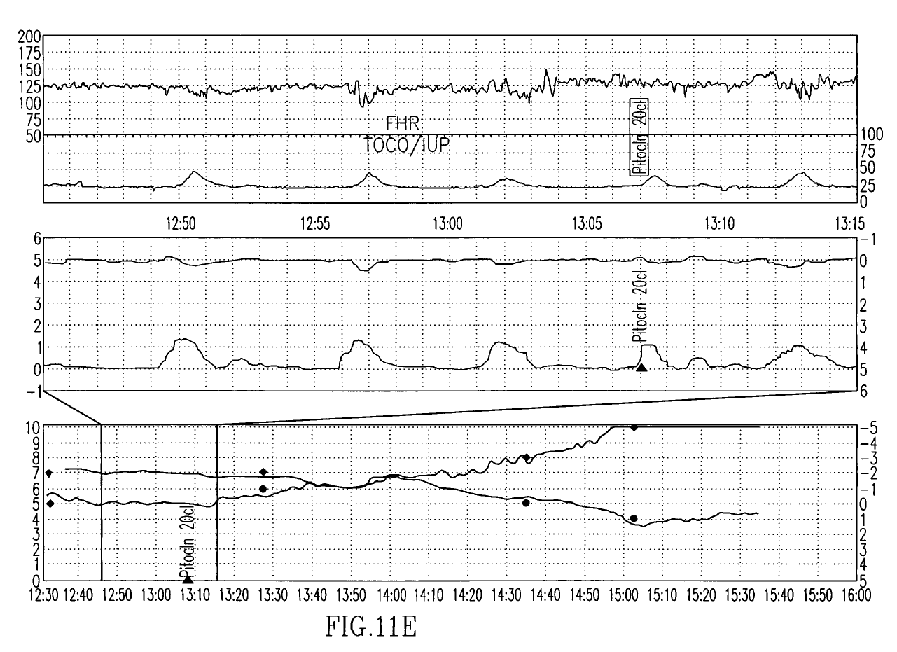

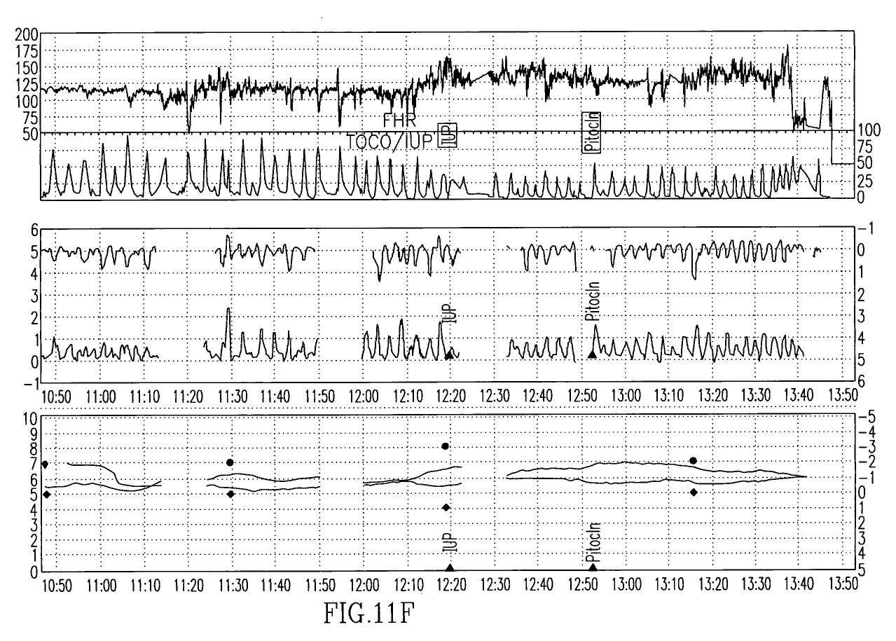

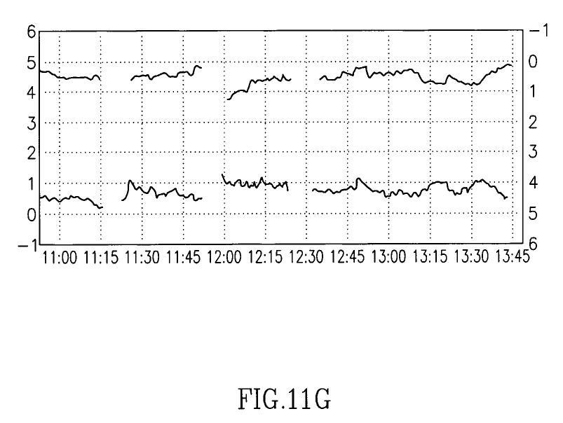

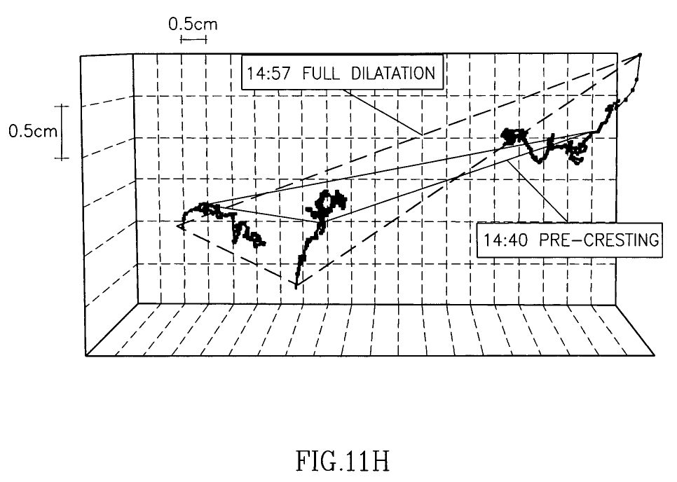

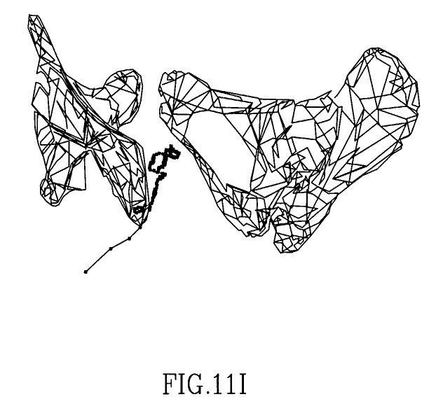

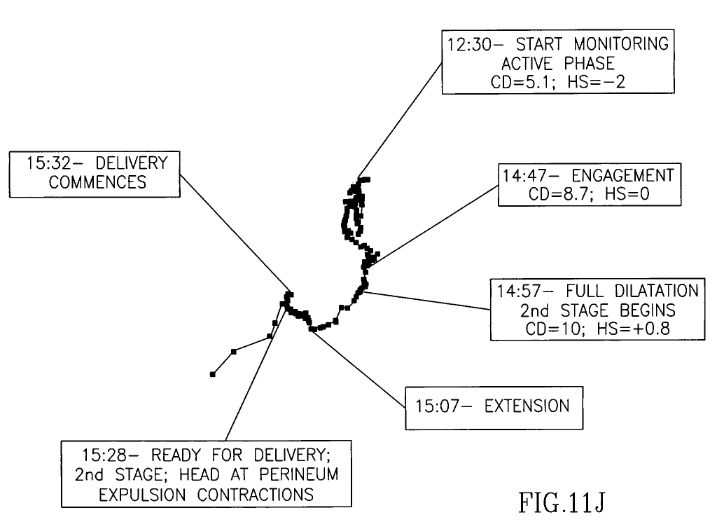

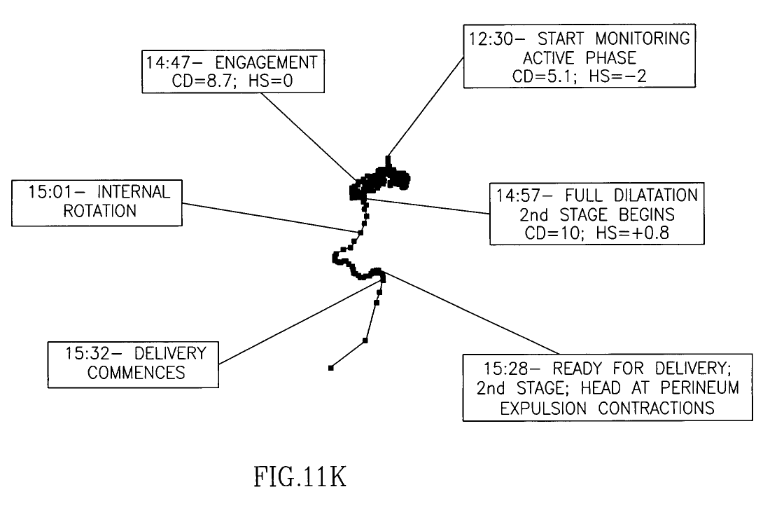

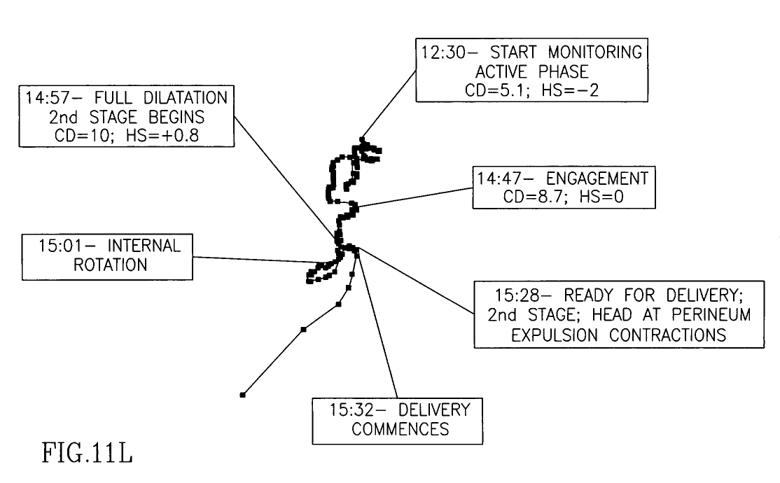

An exemplary representation using various reference frames is a “center of mass” of two or more probes. Optionally, one of the probes is centered, for example the fetal head probe. This type of presentation may serve to reduce noise, for example motion artifact noise. Such a display is described below with respect to FIG. 11H.

Optionally, pelvimetry, for example, using a position sensor or using a different imaging system, such as ultrasound, MRI or CT, is used to generate a geometrical representation of the bones and/or birth canal. Optionally, an operator can define expected changes in this structure during a birth process. Optionally, head station and/or orientation information is shown on such a geometrical representation and/or on an abstract representation, such as a line that is marked with anatomical landmarks and represents the expected path of the fetus.

Optionally, system 100 also attempts to ascertain if there is a geometrical fitting problem, for example, using an estimate of fetal head size determined by the cervical dilatation measurement, and maternal measurements acquired using medical imaging methods.

### Time Estimation

Optionally, system 100 can provide an estimation of time to a future state or to birth (or readiness to birth), where more caregiver attention is required. In one example, if the current birth matches a known pattern, the known pattern is used to estimate time to reach a 2nd stage of labor. Optionally, a neural network or other learning system is used to obtain such an estimate. In another example, an estimate of the next state start time is provided by such a mechanism. In another example, efficacy measurements are used to estimate a length of labor. In another example, CD variations and progress are used to assess a rate of cervical dilatation. In another example, at least a fast moving labor and a slow moving labor are distinguished.

Optionally, higher level measures are also used for time estimation, for example, whether or not engagement occurred and/or whether effective contractions are frequent.

### Exemplary System

FIG. 8A (partially described above) is a schematic diagram showing an exemplary mounting of birth monitoring system 100, in accordance with an exemplary embodiment of the invention.

In an exemplary embodiment of the invention, system 100 comprises external controller 101 and an optional external reference sensor 107, for example shown attached to an anterior superior Iliac spines (ASIS), and/or above the pubis (a sensor 106). Various attachment methods may be used, for example a sticker 108 or a strap. Other positions and/or attachment methods may be used as well. Optionally, three sensors are used, to provide triangulation. However, fewer sensors may be used, for example, a single sensor may be used to detect full dilatation and/or head station. Optionally, this sensor can be on the stomach.

An exemplary alternative location for a reference sensor is a sensor 110 attached, for example to a bed to which mother 120 is fixed, for example, attached using a clasp 121. Alternatively, for example sensor 110 may be attached to the small of the back, coupled to the spine, for example using a strap 112.

Optionally, an abdominal reference sensor is used which is mechanically coupled to the ASIS (anterior superior iliac spines), which may be used as a reference in some embodiments as described above. Ultrasound imaging is optionally used to calibrate system 100 for the relative position and/or orientation of the ASIS and ISL. Alternatively or additionally, calibration is provided using sensor readings, for example, based on an initial head station reading. An exemplary set of sensors is described in PCT/IL2004/001092 filed Nov. 29, 2004, the disclosure of which is incorporated herein by reference.

As noted above, the beginning of the second stage of birth may be determined in various ways. In one example, the head descent (309) is determined by comparing the relative position of fetal head and Cervical os lips 303 and 304. What can be seen is retraction of the Cervical os, once BPD is passed. A potential advantage of this method is that it is a direct measurement of the relative motion of interest (the cresting). Another potential advantage is that maternal motions are not expected to affect this measurement. In another example, the position of the cervix is determined by comparing the positions of the cervical sensors (102, 104) to a plane defined by the Ischial spines (e.g., using sensors on the anterior superior iliac spines) and the fetal head sensor (105). Alternatively, the ASIS spines are used as a reference. Another method is to note if a decrease in variability of CD is not accompanied by a decrease in HS variability.

Optionally, system 100 includes an inclination sensor, or other sensor which indicates the position of mother 120 as a whole and/or a relative position of the abdomen and legs. In an exemplary embodiment of the invention, such an indication can be used to see if the patient moved, thereby changing the relative position of the external transducers on the abdomen, and, as a result, some of the collected information is to be ignored or corrected. Optionally, system 100 is calibrated with information for multiple patient positions, for example by having the patient change position after the sensors are attached and the measurements are used differently based on the inclination or position information. In an exemplary embodiment of the invention, system 100 corrects the calibration so that the values of a previous measurement remain continuous. For example, as it is atypical that cervical dilatation grows 2 cm in 10 seconds and not during a contraction, especially in association with positional change, the calibration of the sensors is changed so that the new dilatation is the same as 20 seconds ago.

In an exemplary embodiment of the invention, sensors 102, 104 and 105 are wired sensors attached to a control box 109 (e.g. using wires 111 or wireless means), which optionally includes status lights 114. Optionally, the status lights indicate if a sensor is correctly attached, for example, based on a sensed value such as electric impedance, acoustic impedance, mechanical resistance, optical measures, and/or ECG, or using physiological measurements, as noted above. Optionally, a differentiation is made to see if the sensor is attached to maternal or fetal tissue, for example, by measuring ECG (which is different for mother and fetus). Alternatively or additionally to using a wire 111 to attach a sensor to box 109 and/or the rest of system 100, a wireless connection, for example using methods known in the art, may be used.

Optionally, system 100 includes a pharmaceutical pump 103 or an attachment to other medical equipment, to allow controller 101 to control and/or block the activity of such equipment. Optionally, system 100 is used for safety and/or as a decision support system to the caregiver (e.g., by giving advice).

Optionally, system 100 includes a connection to a remote unit 122, for example a unit at a nurse's station or a hospital medical information system. Alternatively, system 100 may be worn by a mother, possibly before arriving at a hospital. Optionally, a cellular telephone or PDA is used as a link to remote unit 122, for example at a hospital, before the patient arrives at the hospital. Alternatively or additionally, a wired connection and/or a local wireless connection may be used.

While position and/or orientation information is desirably obtained by internal and external sensors, in some embodiments of the invention, a medical imaging device, such as an ultrasound imager, optical imager or an MRI imager are used instead of some or all of the sensors (e.g., the sensors, if any are used, optionally serve as markers).

Further, the sensors used for position determination can be of many types known in the art, for example, optical magnetic, ultrasonic, and/or be, for example, transmitting, receiving, reflecting and/or field modifying.

### Example of Usage

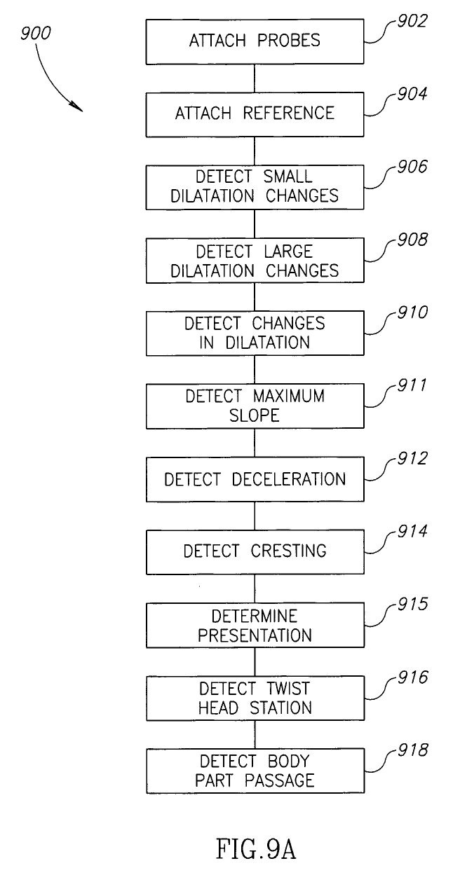

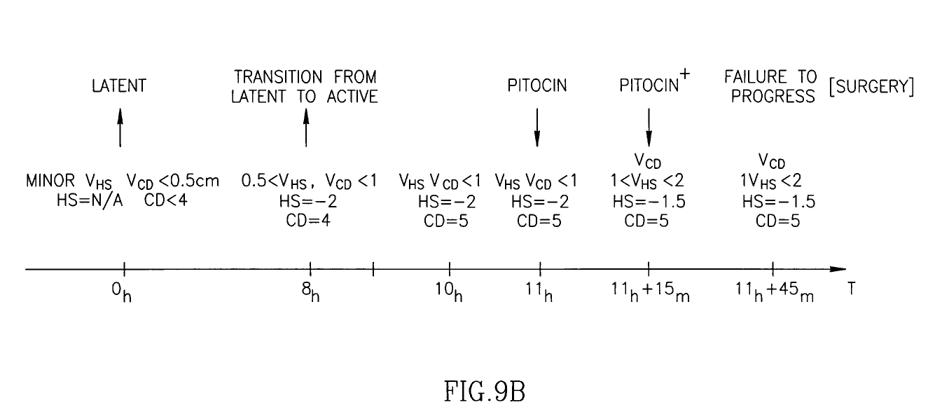

FIG. 9A is a flowchart 900 of automatically identifying one or more states in (normal) labor, in accordance with an exemplary embodiment of the invention.

At 902, one or more position sensing probes are optionally attached to a Cervical os. FIG. 8B is a cross-sectional anatomical view, showing attachment of such sensors 102 and 104 to a Cervical os.

At 904, one or more reference probes/sensors are optionally attached to mother body 120 (106, FIG. 8A) and/or a fetal head 302 (105, FIG. 8A).

At 906, and depending how early in labor the sensors were attached, small dilatation changes and/or head station changes are optionally detected; these may be used to estimate an onset of the latent phase and/or active phase. Additionally, other labor monitoring activities may be provided, at this time and/or during the rest of labor, for example, determining uniformity, mechanical activity and/or strength of contraction and/or monitoring fetal signals. Optionally, the cervical dilatation (CD) changes and head station (HS) changes are combined into a single measure of “contraction activity”.

At 908, the onset of moderate changes in Cervical os diameter, changes in head descent, the relation CD to HS exceeding a critical value and/or increased contraction activity are optionally used to estimate the onset of an acceleration phase (especially as compared to earlier smaller changes in diameter).

At 910, normal labor monitoring of tracking dilatation of the cervix and/or change in head station, is optionally practiced.

At 911, one or more of the following are used to detect a phase of maximum slope: large changes in diameter of the cervix accompanied with large changes in head descent; high contraction activity; and/or that the slope of the relation CD to HS is steady and exceeding a certain value. In an exemplary embodiment of the invention, during the phase of maximum slope, a monitoring is kept to determine that progress is regular. If regular progress stops and there is no indication of approaching full dilatation, an abnormal state may be detected.

At 912, a deceleration phase is detected. Optionally, this is detected by determining a slowing of dilatation rate; a sharp decrease in the variability of cervix diameter not accompanied with a decrease in the variability of changes in head station; and/or a sharp decrease in the relation CD to HS. Alternatively or additionally, changes in internal geometry, such as movement of cervical lips 303 and 304, as described above, may be used for such determining.

At 914, full dilatation and a transition to a second stage of labor is optionally detected by the Cervical os cresting over the fetal head; further increase in changes in head descent during contractions; and/or changes in other coordinates of fetal sensor 105. In some embodiments a variability of ±1 cm and more in HS and other parameters is used as a characteristic of this transition.

At 915, fetal presentation is optionally determined, for example manually, from orientation sensor information or using an imager. This may be used to modify the expectations in later state.

At 916, the engagement, internal rotation, and further extension of the fetal head are optionally determined based on fetal head orientation, rather than solely on actual distance traveled or position relative to a body structure. Optionally, the determined stations are compared to an expected position and/or orientation of the fetal head at the station, as estimated prior to labor or earlier in labor.

At 918, the passage of one or more further body parts through the Cervical os is optionally determined based on changes in cervical dilatation.

In an exemplary embodiment of the invention, when a normal or abnormal state is detected, a set of expected future states is determined and displayed to an operator. In this manner, an estimation of time and/or complexity and/or required equipment may be provided.

In an exemplary embodiment of the invention, when an operator plans on applying a treatment protocol, the system changes the expected measurements and/or states, for example expected duty factor, expected rise time and/or amplitude of a contraction. Optionally, the system tracks the treatment and generates an indication, for example when a determination of effectiveness is to be made and/or when lack of progress or an abnormal situation is detected (e.g., automatically). Optionally, data from a patient is streamed in real-time to a location outside of the room, department and/or hospital where the patient is located and used for telemedicine diagnosis, monitoring and/or provision of a second opinion. Optionally, this data is streamed to a regular treating physician, so he can estimate a time to arrive.

### Abnormal States

Various abnormal states can be detected using a state based approach, some of which have been described above.

In one example, failure to progress of the fetal head, can be optionally observed by large changes in CD not accompanied by head movements during contraction. In general, the mismatch of head station changes and/or advance in response to contractions which changes cervical dilatation, may be viewed as suspicious. Also, a mismatch between CD variability (Vcd) and HS variability (Vhs), or head station progress without CD variations, may be considered suspicious. In an exemplary embodiment of the invention, such mismatch is determined after a short period of time, for example, 20 minutes, 10 minutes or less.

In another example, Vcd and Vhs which are significant but with no net HS & CD progress, may indicate a failure to progress.

In another example, TOCO and/or IUP which are significant with minor or no Vcd or Vhs, may indicate poor efficacy, so drug-based regulation may be indicated.

In another example, changes in cervix dilatation; in head station; and/or in TOCO reading are used to define standards for adequate/effective uterine contractions. Detection of inadequate contractions is optionally used to assist in diagnosing disorders such as baby arrest.

In an exemplary embodiment of the invention, a correlation or mis-correlation of physiological measurement information and information collected by system 100 is used to detect abnormal states. In one example, the phase of HS and/or change (e.g. rise time, fall time, duration etc.) during contraction lags behind or appears before other events (TOCO, IUP, or each other). In another example, changes in CD and HS during contraction are correlated with FHR to determine a cause of bradycardia.

A particular feature of some embodiments of the invention is the generation of an indication that labor s progressing normally, for example, based on changes in CD.

FIG. 9B shows a time line of a problematic birth. At 0 hours, a latent state is present. At 8 hours, an active stage is detected. At 10 hours head station did not advance. At 11 hours Pitocin was administered, when head station still did not advance and variations remained small. At 11.25 hours, more Pitocin was administered, while variations increased and CD did not change. At 11.75 hours, HS and CD did not progress and failure to progress was diagnosed so the patient should now be sent to surgery. In a “standard” protocol, a physician would wait another few hours to determine failure to progress, as no clear indication of lack of effectiveness of the drug would be available.

### Experimental Results

FIGS. 11A-11G show traces and analysis for a labor case monitored in accordance with an exemplary embodiment of the invention.

FIG. 11A shows a personalized partogram 1100 according to an exemplary embodiment of the invention, in a normal birth. Reference 1102 indicates head station. Reference 1104 indicates “corrected” cervical dilatation. Reference 1106 indicates actual cervical dilatation. Reference 1108 indicates short-term average over 20 seconds of actual (not second by second) cervical dilatation measurements, rather than an average as indicated by 1104 and 1106. Reference 1110 indicates short term average over 20 seconds rather than average head station measurements. Reference 1112 and similar diamond markers indicate actual manual measurements of head station and reference 1114 and similar circle markers indicate actual manual measurements of cervical dilatation. The triangular marker indicates manual calibration of head station at start of process.

FIG. 11B shows exemplary traces for a real birth that proceeded normally. A graph 1120 includes FHR in an upper trace and TOCO/IUP in a lower trace. The left scale is a fetal heart rate ranging between 50 and 200 BPM. The right scale is in mmHg (for IUP) and dimensionless for TOCO. An administration of Pitocin is shown at 13:07. A graph 1122 shows, in an upper trace HS changes and in a lower trace CD changes. After full dilatation, the CD changes are set to zero. Here, the left scale is for the change in CD in cm and the left scale is for the change in HS in cm. A graph 1124 shows in an upper trace thereof a progression of HS and in a lower trace thereof a progression of CD. Once full dilatation is reached, CD is clamped to 10 cm. Here, the left scale is for CD in cm and the right scale is for HS in cm. This graph corresponds to FIG. 11A. It should be noted that some sharp variations, due to artifacts, visible in FIG. 11A have been filtered out in graph 1122, as described above.

FIG. 11C shows RMS values of the variations. As can be seen, head station variations (1130) increase gradually and cervical dilatation variations (1132) increase in a stepped profile, as shown in FIG. 4A.

FIG. 11D shows variability calculated using an alternative method described above, and in which HS variabilities are inverted relative to FIG. 11C.

Referring back to FIG. 11B, while not shown, state information, such as current states and expected and/or allowed values may be shown on the trace. A moving window indicating the current time is optionally provided.

FIG. 11E is an enlargement of a 30 minute temporal section of FIG. 11B (also indicated in the bottom trace of FIG. 11E), in which the details (e.g., rise rate and decline rate of CD and HS) can be seen. A preferred display is close to 1 cm/minutes speed of strip chart commonly used in the labor ward. A strong grid line is optionally shown each 5 minutes and every minute a weak grid line.

Referring back to FIG. 11B, at admission time, at which cervical dilatation is low (5 cm) and head station is low (−2 cm). As can be seen, contractions are far apart. Some head movement can be seen in association with some contractions. While contractions are rather far apart, it can be seen that the time between contractions is not really a plateau in the CD trace (while the TOCO trace is normally thresholded). Increased dilatation and increased contraction frequency and contraction variation can be seen at following times.

With further progression of labor, synchronization between head movements and contractions and also substantially continuous contractive movements, can be seen. Once full dilatation is approached, cervical dilatation variation goes down, while head station variation continues increasing. In the 2nd stage head station variation is further increasing or remaining the same, while a positive head station is shown. A normal birth resulted.

FIG. 11F shows traces similar to that of FIG. 11B, of a birth that resulted in a CS.

Blank spaces are where the patient was not lying on her back so no measurements were taken. As can be seen, there is no progress in either parameter.

Referring to FIG. 11G, which corresponds to FIG. 11D, where variations are shown, it is noted that there is no progression in HS or CS variations. In accordance with an exemplary embodiment of the invention, it would be assumed that regular contractions (e.g., >3 every 10 minutes) with regular HS and CD changes would soon result in a progression of labor. Lack of such progression would be considered to indicate an arrest state and a C-section might be indicated. Due to the regular and effective uterine contractions provision, Pitocin would not be recommended. In practice, TOCO was replaced by IUP at 12:20 and at 12:53 Pitocin was administered. The Pitocin did not affect the progress of the labor and possibly caused the hypertonos (or tetanus) which is evident by IUP. When fetal distress was detected, a C-section was carried out.

Use of the methods described above would possibly have clarified (a) within a short period of about 10 or 30 minutes lack of progress would be apparent, giving time to attempt to determine the underlying cause; and/or (b) no drugs need be administered, except possibly to slow down labor.

FIGS. 11H-11L shows three dimensional displays of the data from FIGS. 11A-11E.

FIG. 11H is a center of gravity 3D display showing the relative positions of three sensors: two cervical sensors and one head sensor (in the middle). The display is centered on the center of gravity of the three sensors. Also shown are two triangles each connecting points that are at a same time frame. One triangle connects points at a pre-cresting state and the other shows points at a full dilatation state. The significant motion of the cervix relative to fetal head is clear in this display. This motion can be compared to the relatively small amount of motion up to full dilatation.

FIG. 11I is a perspective view showing the motion of a head sensor during birth, with reference to a schematic (not correct for the patient) pelvis display.

FIG. 11J is a side view (side of patient) showing the correspondence between head positions and states. The amount of motion and change in vector can be clearly seen for several of the states.

FIG. 11K corresponds to FIG. 11J and is a top view.

FIG. 11L corresponds to FIG. 11J and is a front view.

### Additional Exemplary Uses of System

System 100 may also have other uses, for example, a less complete use than monitoring an entire birth. For example, generating an alert when a midwife is required, warning a doctor that a birth is imminent (e.g., the labor progress matches a “fast track” labor).

Optionally, system 100 is used during semi-invasive procedures, such as vacuum assisted birth or forceps assisted birth, in which the existence, magnitude and/or effect of a contraction (e.g., fetal head vector) can be provided to an operator to help the operator work with the contraction and/or avoid interference, for example.

While the description has focused on head-first birth, optionally, the system is used to monitor a breech birth (e.g., with associated changes in the expected parameter values and/or states).

Optionally, system 100 is used to support shift changes and/or presentation of a case to an expert. In one example, the system can show a history. In another example, the system can answer various questions posed by such an expert, for example, regarding progress. Alternatively or additionally, the system can function as a decision support system that shows suggestion and/or shows analyzed information. Optionally, the system differentiates between information (internally generated or manually inputted) that has been authorized by an operator and information which has not been authorized.

In an exemplary embodiment of the invention, system 100 provides a reliable method of documentation and authentication by the attending staff. Optionally, the data is digitally stored by the system and/or securely communicated and stored by site IT administrator reliably for a period of 21 years. Optionally, the data is encrypted to maintain patient privacy. Authentication methods known in the art may be provided for allowing annotation and/or viewing of the stored data. Templates and ranges generated from the data are optionally openly available and do not include patient identifying information, but may include other information, such as demographic information.

### Specialized Devices

The above description has focused on a general purpose device. However, the present invention also encompasses, in some embodiments thereof, more dedicated devices.

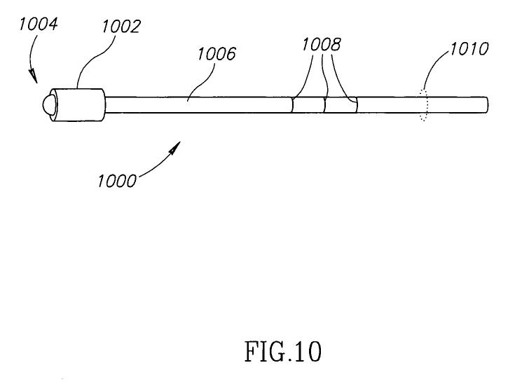

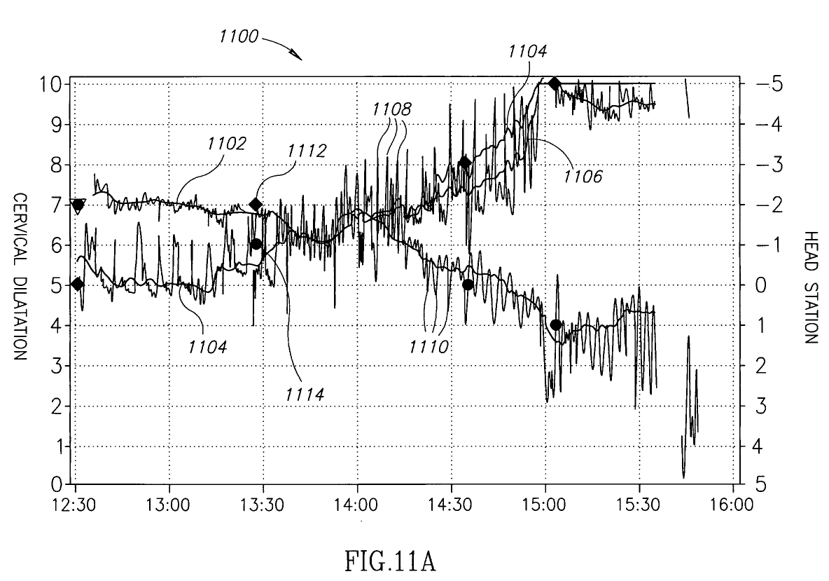

FIG. 10 is a schematic illustration of a second stage detection device 1000, in accordance with an exemplary embodiment of the invention. Device 1000 includes a head section 1002 including an anchor 1004 for attachment to the lips of a Cervical os (e.g., like sensors 102, 104). Device 1100 also includes a body 1106 which is long enough to reach out of a vagina (e.g., 20 cm or more) and includes a plurality of markings 1108.

Cresting of fetal head 302 will cause a retrograde motion of device 1000, causing one or more of the markings 1008 to disappear into the birth canal. Optionally, a placeholder 1010 (shown dashed) is provided on body 1006, so that an exact number of markings visible need not be remembered.

In a particular implementation, a fetal head electrode is used, with suitable markings and/or a placeholder. Optionally, body 1006 is made stiff enough so that it will not fold inside the birth canal but remain substantially straight (e.g., along the axis of the birth canal).

By providing two such devices 1000, asymmetrical cresting can be detected.

Optionally, an audible alarm is provided in device 1000, for example based on closing of electrical contacts or changes in impedance. Optionally, the proximal part of body 1006 is attachable to the patient, for example, to her thigh, so that it does not move axially (or generates an alarm when it moves axially).

Optionally, a single device is used to show both entry into 2nd stage and other information, for example head rotation or bending, for example, by including suitable sensors in body 1006 which sense bending or rotation thereof.

While the above description has focused on human birth, optionally, a similar system is used for animal husbandry, for example, for tracking birth of animals that are out in a field (e.g., cows) or for tracking the birth of expensive animals, such as thoroughbred horses. The numbers and/or sizes may vary for non-human animals. Optionally, a wireless halter is used to communicate with implanted sensors and with a remote base. The halter may be attached by wire or wireless means to the internal sensors.

It will be appreciated that the above described methods of labor management and monitoring may be varied in many ways, including, changing the order of steps and the types of sensors used. In addition, a multiplicity of various features, both of method and of devices have been described. In some embodiments mainly methods are described; however, also apparatus adapted for performing the methods are considered to be within the scope of the invention. It should be appreciated that different features may be combined in different ways. In particular, not all the features shown above in a particular embodiment are necessary in every similar embodiment of the invention. Further, combinations of the above features are also considered to be within the scope of some embodiments of the invention. Also within the scope of the invention are kits which include sets of medical devices suitable for performing a single or a small number of measurements. Also, within the scope are hardware, software and computer readable-media including such software and/or other means (e.g., standard computers, ASICs, other hardware, software, circuitry, analog devices, digital devices, firmware) which is used for carrying out and/or guiding the steps described herein, such as signal processing and decision support. In particular a controller may be configured for (e.g., manufactured for or programmed for or otherwise adapted for) carrying out the methods. Section headings are provided for assistance in navigation and should not be considered as necessarily limiting the contents of the section. When used in the following claims, the terms “comprises”, “includes”, “have” and their conjugates mean “including but not limited to”.

It will be appreciated by a person skilled in the art that the present invention is not limited by what has thus far been described. Rather, the scope of the present invention is limited only by the following claims.

1. A method of monitoring a birth process, comprising:
receiving, over time, a plurality of position signals from one or more positioning elements or tissue areas located at least one of a cervix and a fetal head; and
determining a discrete state of labor of a fetus that is wholly inside a body responsive to said position signals, with a temporal resolution of better than 15 minutes, said discrete state being other than a start or stop of labor and encompassing more than a single contraction, said state including a state other than an abnormal fetal head position.

2. A method according to claim 1, wherein said one or more positioning elements comprises a wireless transponder.

3. A method according to claim 1, wherein receiving comprises receiving from one or more tissue areas identifiable using an imaging system.

4. A method according to claim 1, wherein receiving comprises receiving from at least one positioning element.

5. A method according to claim 1, wherein said one or more positioning elements comprises a transmitter.

6. A method according to claim 1, wherein said one or more positioning elements comprises a marker.

7. A method according to claim 1, wherein said discrete state comprises at least one state from a list of states including: failure to progress, inefficient uterine contractions, onset of active labor, full dilatation, optimal uterine activity, individual maximum slope of dilatation, fetal head internal rotation, fetal head extension, pre-cresting, arrest disorder, canal arrest, abnormal expulsion contractions, normal expulsion contractions, efficacy of drug administration and readiness for delivery.

8. A method according to claim 7, comprising determining at least 2 states from said list at different times.

9. A method according to claim 7, comprising determining at least 4 states from said list at different times.

10. A method according to claim 7, comprising determining at least 6 states from said list at different times.

11. A method according to claim 1, wherein the position signals comprises fetal head position signals and cervical OS position signals.

12. A method according to claim 1, wherein the position signals do not comprise absolute cervical dilatation signals.

13. A method according to claim 1, wherein the position signals comprise absolute cervical dilatation signals.

14. A method according to claim 13, comprising modifying the cervical dilatation signals to reflect a scale on which full dilatation is 10 cm.

15. A method according to claim 1, wherein determining comprises determining based on an analysis of short term changes in said signals, within a time period of a contraction cycle.

16. A method according to claim 15, wherein said analysis comprises an analysis of changes in a fetal head position.

17. A method according to claim 16, wherein said analysis comprises an analysis of a spatial vector of fetal head motion.

18. A method according to claim 15, wherein said analysis comprises an analysis of changes in cervical geometry.

19. A method according to claim 15, wherein said analysis comprises an analysis of rate of change of a position.

20. A method according to claim 15, wherein said analysis comprises an analysis over a plurality of contractions.

21. A method according to claim 13, wherein said determining comprises determining based on a duty factor of a plurality of contractions.

22. A method according to claim 1, wherein said determining comprises determining that a labor is progressing normally.

23. A method according to claim 1, wherein said determining comprises determining that a labor is progressing abnormally.

24. A method according to claim 1, wherein said determining comprises determining a type of contraction.

25. A method according to claim 1, wherein said determining is based on non-geometrical physiological signals of at least one of mother and fetus.

26. A method according to claim 25, wherein said determining comprises analyzing a phase delay between non-geometric physiological and geometrical measurements.

27. A method according to claim 25, wherein said physiological signals comprise pressure signals.

28. A method according to claim 25, wherein said physiological signals comprise EMG signals.

29. A method according to claim 25, wherein said physiological signals comprise heart rate signals.

30. A method according to claim 1, wherein determining comprises determining a state on a personalized time/progression scale.

31. A method according to claim 1, comprising matching a progression of labor to one of a plurality of templates.

32. A method according to claim 1, comprising estimating a time to reach a future state, based on said signals.

33. A method according to claim 1, wherein said position signals are acquired using a reference remote from said elements.

34. A method according to claim 1, comprising determining at least one of an orientation change and magnitude change in a vector of a fetal head.

35. A method according to claim 34, wherein said change in vector comprises a change in orientation of a fetal head.

36. A method according to claim 34, comprising generating a head station value indicating the spatial progression of the fetal head in a birth canal.

37. A method according to claim 34, wherein said vector comprises a vector of motion of said head during a contraction.

38. A method according to claim 37, comprising comparing said vector to an expected head path in a maternal body.

39. A method according to claim 37, comprising determining an asymmetry between forward motion and backward motion of said head.

40. A method of labor management, comprising:
(a) collecting information about a labor process;
(b) generating a personalized progression representation based on said information;
(c) identifying a relationship between a parameter of said representation and a norm, within 20 minutes of said parameter changing its relationship relative to the norm; and
(d) selectively modifying a treatment of the labor responsive to said identification.

41. A method according to claim 40, wherein said identifying comprises identifying by computer circuitry.

42. A method according to claim 40, comprising suggesting a modification by computer circuitry.

43. A method according to claim 40, wherein identifying comprises identifying that said parameter is outside a norm.

44. A method according to claim 40, wherein identifying comprises identifying that said parameter is inside a norm.

45. A method according to claim 40, wherein selectively modifying comprises not modifying.

46. A method according to claim 40, wherein generating said personalized progression representation comprises statistical analysis of said collected information.

47. A method according to claim 46, wherein said statistical analysis comprises long term analysis.

48. A method according to claim 46, wherein said statistical analysis comprises short-term analysis.

49. A method according to claim 46, wherein said statistical analysis comprises generating a histogram.

50. A method according to claim 40, wherein said personalized progression representation includes an expected rate of change.

51. A method according to claim 40, wherein said personalized progression representation includes an identification of at least three labor states.

52. A method according to claim 40, wherein said personalized progression representation comprises an indication that an individual maximum slope is about to be achieved.

53. A method according to claim 52, wherein said indication comprises a dedicated display.

54. A method according to claim 40, wherein said indication comprises a state display including a presentation of states according to their relative context and including a history of states.

55. A method according to claim 40, wherein said indication comprises a display of individual maximum slope.

56. A method of monitoring a labor process, comprising:
receiving, over time, a plurality of positional information from one or more positioning elements or tissue segments located at least one of a cervix and a fetal head;
determining at least one change in magnitude of positional information within a contraction;
analyzing said at least one change; and
determining a status of said labor based on said analysis.

57-141. (canceled)
